(19) United States
(12) Patent Application Publication
(10) Pub. No.: US 2017/0216189 A1
(43) Pub. Date: Aug. 3, 2017

(54) POLYURETHANE UREA SOLUTIONS FOR COSMETIC COMPOSITIONS

(71) Applicant: Covestro Deutschland AG, Leverkusen (DE)

(72) Inventors: Sebastian Dörr, Düsseldorf (DE); Sophie Viala, Köln (DE); Paula Cristina Alves Rodrigues, Düsseldorf (DE)

(21) Appl. No.: 15/501,039

(22) PCT Filed: Mar. 26, 2015
(86) PCT No.: PCT/EP2015/056579

§ 371 Date: Feb. 1, 2017

(30) Foreign Application Priority Data
Aug. 5, 2014 (EP) 14179782.9

(51) Int. Cl. A61K 8/87; A61K 8/86; A61K 8/40; A61K 8/41; A61Q 17/04; A61K 8/34

(52) U.S. Cl. CPC A61K 8/87; A61Q 17/04; A61K 8/34; A61K 8/40; A61K 8/41; A61K 8/86

(57) ABSTRACT

The invention relates to a method for producing a cosmetic composition, comprising at least one polyurethane urea which does not contain any ionically hydrophilizing groups and is dissolved in a solvent or solvent mixture, wherein the solvent consists of one or more monohydroxy-functional alcohol(s), or a solvent mixture that consists of organic solvents and contains ≧50 wt. %, relative to the total weight of the solvent mixture, of at least one monohydroxy-functional alcohol. The invention also relates to a cosmetic composition obtainable according to the method of the invention as well as to a method for forming a cosmetic coating on skin, nails and/or keratin fibers using the cosmetic compositions of the invention. The invention further relates to a sunscreen composition containing a polyurethane urea as well as to said sunscreen composition for protecting skin and/or hair from negative effects of sun rays.

### CROSS-REFERENCE TO RELATED APPLICATIONS

This Application is a National Phase Application of PCT/EP2015/056579 Mar. 26, 2015, which claims priority to European Application No. 14179782.9, filed Aug. 5, 2014, both of which are incorporated herein by reference.

### FIELD OF THE INVENTION

The present invention relates to a process for producing a cosmetic composition using polyurethane ureas, and to a cosmetic composition obtainable by the process of the invention and to a process for producing a cosmetic coating on skin, nails and/or keratinic fibers using the cosmetic compositions of the invention. The invention also relates to a sunscreen composition comprising a polyurethane urea and to this sunscreen composition for protection of skin and/or hair from adverse effects of solar radiation.

### BACKGROUND OF THE INVENTION

Cosmetic products based mainly on readily evaporating alcohols, for example ethanol, are enjoying increasing popularity on the market. The rapid evaporation of the alcohol results in rapid drying of the compositions after application to the body, and so they are rapidly ready for use. Moreover, especially products for application to the skin leave a freshening and cooling sensory impression and also exhibit only very low tackiness of the film formed on the skin. Alcohol-based compositions are preferably also used for production of transparent sun sprays or gels. Since transparent sprays and gels should not contain any visible UV-absorbing particles, however, there is a restriction in the selection of the UV filters in such sunscreen compositions and a multitude of oil-soluble UV filters are used. Alcohol-based transparent sun sprays are described, for example, in WO 2007/068699 A1 and DE 20 2010 006 005 U1.

Cosmetic products for application to the body usually contain a polymer-based film former. Known good film-forming polymers for cosmetic compositions include aqueous dispersions of polyurethane ureas, as described, for example, in WO 2009/118105 A1, WO 2009/118103 A1, WO 2011/107462 A1 WO 2012/130683 A1 and WO 2014/095164 A1. Cosmetic compositions comprising the aqueous polyurethane urea dispersions described have some advantages, for example a high SPF-boosting effect (boosting effect on the sun protection factor) in sunscreen compositions, and hence a reduced amount of sunscreen filter substances that have to be used in order to obtain a particular high SPF (sun protection factor). However, the known aqueous dispersions of the polyurethane ureas have some disadvantages in cosmetic compositions based predominantly on alcoholic solvents. They lead, for example, to turbidity in the cosmetic products, especially in those products containing UV filters that are exclusively oil-soluble. This is perceived as being disadvantageous for many applications. The additional proportion of water which is additionally introduced into the product by the aqueous dispersions can additionally result in elevated tackiness and a prolonged drying time of the cosmetic products. Another disadvantage is that the polyurethane ureas according to prior art have hydrophilizing groups, especially ionically hydrophilizing groups, which are introduced into the polymers by means of costly compounds that bear these groups.

Polyurethane ureas that bear ionically hydrophilizing groups additionally do not generally form clear solutions in alcohols, which means that they are not very suitable for use in transparent cosmetic compositions.

Polyurethane film formers that bear such hydrophilizing groups, especially ironically hydrophilizing groups, are also described, for example, in DE 4241118 A1.

It is common knowledge that polyurethane ureas, because of their structure, tend to precipitate or crystallize out of organic solutions. It is therefore problematic to produce organic solutions of polyurethane ureas having a sufficiently high molecular weight without precipitation of the polyurethane ureas out of the solvents, and therefore no clear, storage-stable solutions are obtained.

For prevention of this crystallization, solvent mixtures comprising solvents which are now counted among the potentially harmful solvents on the basis of growing toxicological knowledge are recommended, for example toluene or xylene.

However, the use of such co-solvents for polyurethane ureas for use in cosmetic products is not possible merely for approval-related legal reasons. Moreover, the film formers used in WO 2007/068699 A1 and DE 20 2010 006 005 U1, by contrast with polyurethane urea dispersions, have only a low SPF boosting effect, and so the use of high amounts of sunscreen filter substances is required in order to achieve a high SPF.

### SUMMARY OF THE INVENTION

The present invention provides a process for producing cosmetic compositions, which enables use of polyurethane ureas as film formers evening alcoholic cosmetic formulations, especially those containing oil-soluble UV filters, without the latter having troublesome turbidity or necessarily containing an elevated water content. The cosmetic formulations obtained should nevertheless have the advantages known from the state of the art of aqueous polyurethane urea dispersions in cosmetic formulations, for example an SPF-boosting effect in sunscreen compositions. The cosmetic composition produced should therefore be suitable for treatment of nails, skin and/or keratinic fibers, more preferably for treatment of skin and/or hair.

These and other advantages and benefits of the present invention will be apparent from the Detailed Description of the Invention herein below

### DETAILED DESCRIPTION OF THE INVENTION

The present invention provides a process for producing a cosmetic composition, characterized in that at least one polyurethane urea which has no ionically hydrophilizing groups and has been dissolved in a solvent or solvent mixture is used, the solvent consisting of one or more monohydroxy-functional alcohols or being a solvent mixture consisting of organic solvents and containing ≧50% by weight, based on the total mass of the solvent mixture, of at least one monohydroxy-functional alcohol. The polyurethane urea used is formed from
a) at least one of an aliphatic, an araliphatic and a cycloaliphatic diisocyanate,
b) at least one polyether polyol having a number-average molecular weight Mₙ of ≧400 and ≦6000 g/mol and a hydroxyl functionality of ≧1.5 and ≦4,
c) at least one amino-functional compound having at least two isocyanate-reactive amino groups,
d) optionally, at least one alcohol having at least two hydroxyl groups and a molar mass of ≧60 and ≦399 g/mol,
e) optionally, at least one compound having a group reactive toward isocyanate groups and
f) optionally, ≦20% by weight, based on the total mass of the polyurethane urea, of at least one different polyol than b) having a number-average molecular weight Mₙ of ≧500 and ≦6000 g/mol and a hydroxyl functionality of ≧1.5 and ≦4.

It has been found that, surprisingly, it is possible by the process of the invention to provide alcoholic cosmetic compositions containing polyurethane ureas as film formers, without the cosmetic compositions obtained having troublesome turbidity or containing elevated water content. Cosmetic formulations containing UV filters dissolved in oil do not exhibit any troublesome turbidity either. The cosmetic formulations nevertheless have the advantages known from the state of the art of aqueous polyurethane urea dispersions in cosmetic formulations, for example an SPF-boosting effect in sunscreen compositions.

The invention further provides for the use of a polyurethane urea which has no ionically hydrophilizing groups and has been dissolved in a solvent or solvent mixture, wherein the solvent consists of one or more monohydroxy-functional alcohols or a solvent mixture consisting of organic solvents and containing ≧50% by weight, based on the total mass of the solvent mixture, of at least one monohydroxy-functional alcohol, for the production of a cosmetic composition.

The invention further provides for the use of a polyurethane urea which has no ionically hydrophilizing groups and has been dissolved in a solvent or solvent mixture, wherein the solvent consists of one or more monohydroxy-functional alcohols or a solvent mixture consisting of organic solvents and containing ≧50% by weight, based on the total mass of the solvent mixture, of at least one monohydroxy-functional alcohol, for cosmetic coating of nails, skin and/or keratinic fibers, preferably of skin and/or hair, more preferably of skin.

The polyurethane urea used is formed from
a) at least one of an aliphatic, an araliphatic and a cycloaliphatic diisocyanate,
b) at least one polyether polyol having a number-average molecular weight Mₙ of ≧400 and ≦6000 g/mol and a hydroxyl functionality of ≧1.5 and ≦4,
c) at least one amino-functional compound having at least two isocyanate-reactive amino groups,
d) optionally, at least one alcohol having at least two hydroxyl groups and a molar mass of ≧60 and ≦399 g/mol,
e) optionally, at least one compound having a group reactive toward isocyanate groups and
f) optionally, ≦20% by weight, based on the total mass of the polyurethane urea, of at least one different polyol than b) having a number-average molecular weight Mₙ of ≧500 and ≦6000 g/mol and a hydroxyl functionality of ≧1.5 and ≦4.

The dissolved polyurethane urea used in accordance with the invention, including the solvent or solvent mixture, is also referred to hereinafter as polyurethane urea solution.

“Dissolved” in the context of the invention means clear liquid mixtures of at least two substances that are homogeneous and monophasic at 23° C. “Clear” in the context of the present invention means that the turbidity values of the solution are ≦200 NTU (Nephelometric Turbidity Unit), preferably ≦50 NTU, more preferably ≦10 NTU and most preferably ≦3 NTU. Turbidity values are determined by a scattered light measurement at a 90° angle (nephelometry) at a measurement radiation wavelength of 860 nm in accordance with DIN EN ISO 7027, conducted at 23° C. with a model 2100AN laboratory turbidimeter from HACH LANGE GmbH, Berlin, Germany.

Polyurethane ureas in the context of the invention are polymeric compounds having at least two, preferably at least three, urethane-containing repeat units

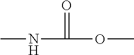

and additionally also urea-containing repeat units:

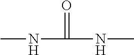

Ionically hydrophilizing groups in the context of the invention are those which could be introduced into the polyurethane urea, for example, by means of suitable anionically or potentially anionically hydrophilizing compounds having at least one isocyanate-reactive group, such as a hydroxyl or amino group, and at least one functionality, for example, —COO-M⁺, —SO₃-M⁺, —PO(O-M⁺)₂ where M⁺, for example is a metal cation, H⁺, NH₄⁺, NHR₃⁺ where each R is a C₁-C₁₂-alkyl radical, C₅-C₆-cycloalkyl radical and/or a C₂-C₄-hydroxyalkyl radical, which enters into a pH-dependent dissociation equilibrium on interaction with aqueous media and in this way may be negatively charged or uncharged.

Suitable anionically or potentially anionically hydrophilizing compounds are mono- and dihydroxycarboxylic acids, mono- and dihydroxysulfonic acids, and mono- and dihydroxyphosphonic acids and salts thereof. Examples of such anionic or potentially anionic hydrophilizing agents are dimethylolpropionic acid, dimethylolbutyric acid, hydroxypivalic acid, malic acid, citric acid, glycolic acid, lactic acid and the propoxylated adduct of 2-butenediol and NaHSO₃, as described in DE-A 2 446 440, pages 5-9, formula I-III.

Potentially anionic (and also generally potentially ionic) groups in the context of this invention are understood to mean those which can be converted by neutralization to an anionic (ionic) group.

In a preferred embodiment of the process of the invention, the polyurethane urea used does not have any hydrophilizing groups, i.e. neither ionic nor nonionic hydrophilizing groups.

Nonionic hydrophilizing groups in the context of the invention are those which could be introduced into the polyurethane urea, for example, by means of suitable nonionically hydrophilizing compounds, for example polyoxyalkylene ethers containing at least one hydroxyl or amino group. Examples are the monohydroxy-functional polyalkylene oxide polyether alcohols having a statistical average of 5 to 70, preferably 7 to 55, ethylene oxide units per molecule, as obtainable in a manner known per se by alkoxylation of suitable starter molecules (described, for example, in Ullmanns Encyclopadie der technischen Chemie [Ullmann's Encyclopedia of Industrial Chemistry], 4th edition, volume 19, Verlag Chemie, Weinheim p. 31-38). These compounds are either pure polyethylene oxide ethers or mixed polyalkylene oxide ethers, in which case, however, they contain at least 30 mol %, preferably at least 40 mol %, based on all alkylene oxide units present, of ethylene oxide units.

The polyurethane ureas of the present invention are used in the process of the invention for producing the cosmetic compositions in dissolved form in a solvent or solvent mixture, and hence as polyurethane urea solutions and not as an aqueous dispersion.

The polyurethane urea used in accordance with the invention has been formed from
a) at least one of an aliphatic, an araliphatic and a cycloaliphatic diisocyanate,
b) at least one polyether polyol having a number-average molecular weight Mₙ of ≧400 and ≦6000 g/mol and a hydroxyl functionality of ≧1.5 and ≦4,
c) at least one amino-functional compound having at least two isocyanate-reactive amino groups,
d) optionally, at least one alcohol having at least two hydroxyl groups and a molar mass of ≧60 and ≦399 g/mol,
e) optionally, at least one compound having a group reactive toward isocyanate groups and
f) optionally, ≦20% by weight, based on the total mass of the polyurethane urea, of at least one different polyol than b) having a number-average molecular weight Mₙ of ≧500 and ≦6000 g/mol and a hydroxyl functionality of ≧1.5 and ≦4.

The number-average molecular weight is always determined in the context of this application by gel permeation chromatography (GPC) in tetrahydrofuran at 23° C. The procedure is according to DIN 55672-1: “Gel permeation chromatography, Part 1—tetrahydrofuran as eluent” (SECurity GPC System from PSS Polymer Service, flow rate 1.0 ml/min; columns: 2×PSS SDV linear M, 8×300 mm, 5 μm; RID detector). Polystyrene samples of known molar mass are used for calibration. The number-average molecular weight is calculated with software support. Baseline points and evaluation limits are fixed in accordance with DIN 55672 Part 1.

Further preferably, the polyurethane urea is formed from ≧5% and ≦60% by weight of component a), ≧30% and ≦90% by weight of component b), ≧2% and ≦25% by weight of component c), ≧0% and ≦10% by weight of component d), ≧0% and ≦10% by weight of component e) and ≧0% and ≦20% by weight of component f), based in each case on the total mass of the polyurethane urea, where components a) to f) add up to 100% by weight.

Especially preferably, the polyurethane ureas is formed from ≧10% and ≦40% by weight of component a), ≧55% and ≦85% by weight of component b), ≧5% and ≦20% by weight of component c), ≧0% and ≦3% by weight of component d), ≧0% and ≦3% by weight of component e) and ≧0% and ≦1% by weight of component f), based in each case on the total mass of the polyurethane urea, where components a) to f) add up to 100% by weight.

Compounds suitable as component a) are, for example, butylene 1,4-diisocyanate, pentamethylene 1,5-diisocyanate (PDI), hexamethylene 1,6-diisocyanate (HDI), isophorone diisocyanate (IPDI), 2,2,4- and/or 2,4,4-trimethylhexamethylene diisocyanate, the isomeric bis(4,4′-isocyanatocyclohexyl)methanes or mixtures thereof with any isomer content (H12-MDI), cyclohexylene 1,4-diisocyanate, 4-isocyanatomethyloctane 1,8-diisocyanate (nonane triisocyanate), 1,3- and/or 1,4-bis(2-isocyanatoprop-2-yl)benzene (TMXDI), 1,3-bis(isocyanatomethyl)benzene (XDI) and alkyl 2,6-diisocyanatohexanoates (lysine diisocyanates) having C₁-C₈-alkyl groups.

As well as the aforementioned polyisocyanates, it is also possible to use proportions of modified diisocyanates or triisocyanates having isocyanurate, urethane, allophanate, biuret, iminooxadiazinedione and/or oxadiazinetrione structure.

Preferably, the polyisocyanates or polyisocyanate mixtures are of the aforementioned type with a mean NCO functionality of ≧2 and ≦4, preferably ≧2 and ≦2.6 and more preferably ≧2 and ≦2.4.

Preferably, component a) is selected from an aliphatic, an araliphatic and a cycloaliphatic diisocyanate having at least one isocyanate group bonded to a secondary or tertiary carbon atom.

More preferably, component a) is selected from IPDI and/or H12-MDI.

Further preferably, no aromatic polyisocyanates are used for preparation of the polyurethane urea.

Component a) is preferably used in amounts of ≧5% and ≦60% by weight, more preferably ≧10% and ≦40% by weight and most preferably of ≧15% and ≦35% by weight, based on the total weight of the polyurethane ureas.

Component b) consists of one or more polyether polyols having a number-average molecular weight Mₙ≧400 and ≦6000 g/mol and a hydroxyl functionality of ≧1.5 and ≦4, preferably having a number-average molecular weight Mₙ≧500 and ≦2500 g/mol and a hydroxyl functionality of ≧1.9 and ≦3 and more preferably having a number-average molecular weight Mₙ≧1000 and ≦2000 g/mol and a hydroxyl functionality of ≧1.9 and ≦2.1.

Suitable polyether polyols of component b) are, for example, the poly(tetramethylene glycol) polyether polyols known in polyurethane chemistry, as obtainable by polymerization of tetrahydrofuran by means of cationic ring opening.

Likewise suitable polyether polyols are the addition products of styrene oxide, ethylene oxide, propylene oxide, butylene oxide and/or epichlorohydrin onto di- or polyfunctional starter molecules. Polyalkylene glycols in particular, such as polyethylene glycols, polypropylene glycols and or polybutylene glycols, are applicable, especially with the abovementioned preferred molecular weights. The polyether polyols preferably have a proportion of groups obtained from ethylene oxide of <50% by weight, preferably <30% by weight.

Suitable starter molecules used may be all compounds known according to the state of the art, for example water, butyldiglycol, glycerol, diethylene glycol, trimethylolpropane, propylene glycol, sorbitol, ethylenediamine, triethanolamine, butane-1,4-diol.

Preferably, component b) is selected from polypropylene glycols and/or poly(tetramethylene glycol) polyether polyols, more preferably selected from poly(tetramethylene glycol) polyether polyols.

In a preferred employment of the invention, component b) comprises one or more poly(tetramethylene glycol) polyether polyols having a number-average molecular weight Mₙ≧500 and ≦2500 g/mol and a hydroxyl functionality of ≧1.9 and ≦2.1.

In a particularly preferred embodiment, component b) is a mixture of poly(tetramethylene glycol) polyether polyols I having a number-average molecular weight Mₙ of ≧400 and ≦1500 g/mol, more preferably of ≧600 and ≦1200 g/mol, most preferably of 1000 g/mol, and poly(tetramethylene glycol) polyether polyols II having a number-average molecular weight Mₙ of ≧1500 and ≦8000 g/mol, more preferably of ≧1800 and ≦3000 g/mol, most preferably of 2000 g/mol.

The weight ratio of the poly(tetramethylene glycol) polyether polyols I to the poly(tetramethylene glycol) polyether polyols II is preferably in the range of ≧0.1 and ≦10, more preferably in the range of ≧0.2 and ≦8, most preferably in the range of ≧1 and ≦6.

Component b) is preferably used in amounts of ≧30% and ≦90% by weight, more preferably ≧50% and ≦85% by weight, most preferably of ≧55% and ≦75% by weight, based on the total weight of the polyurethane urea.

Component c) is one or more amino-functional compounds having at least two isocyanate-reactive groups.

Suitable components c) are, for example, di- or polyamines such as ethylene-1,2-diamine, 1,2- and 1,3-diaminopropane, 1,4-diaminobutane, 1,6-diaminohexane, 2,2,4- and 2,4,4-trimethylhexamethylenediamine, 2-methylpentamethylenediamine, diethylenetriamine, triaminononane, xylylene-1,3- and 1,4-diamine, α,α,α′,α′-tetramethylxylylene-1,3- and -1,4-diamine and 4,4′-diaminodicyclohexylmethane (H12-MDA), isophoronediamine (IPDA) and/or 1,2-dimethylethylenediamine.

Preferably, component c) is selected from ethyleneamine, IPDA and H12-MDA, more preferably from isophoronediamine and/or H12-MDA, and component c) is most preferably H12-MDA.

The compounds of component c) preferably do not contain any hydrophilizing groups, and more particularly no ionically or potentially anionically hydrophilizing groups.

In a particularly preferred embodiment of the invention, component c) is selected from amines having at least two isocyanate-reactive amino groups bonded to primary and/or secondary carbon atoms.

Further preferably, component c) is selected from diamines of symmetric structure. Most preferably, component c) is selected from symmetric diamines having at least two amino groups bonded to primary and/or secondary carbon atoms; component c) is especially preferably H12-MDA.

Component c) is preferably used in amounts of ≧2% and ≦25% by weight, more preferably ≧5% and ≦20% by weight and most preferably ≧9% and ≦16% by weight, based on the total weight of the polyurethane urea.

In a preferred embodiment of the invention, either component a) is H12-MDI or component c) is H12-MDA or component a) is H12-MDI and component c) is H12-MDA.

Optionally, the polyurethane urea is additionally formed from component d), one or more alcohols having at least two hydroxyl groups and a molar mass of ≧60 and ≦399 g/mol, for example polyols of the molar mass range mentioned having up to 20 carbon atoms, such as ethylene glycol, diethylene glycol, triethylene glycol, propane-1,2-diol, propane-1,3-diol, butane-1,4-diol, 1,3-butylene glycol, cyclohexanediol, cyclohexane-1,4-dimethanol, hexane-1,6-diol, neopentyl glycol, hydroquinone dihydroxyethyl ether, bisphenol A (2,2-bis(4-hydroxyphenyl)propane), hydrogenated bisphenol A (2,2-bis(4-hydroxyphenyl)propane), trimethylolpropane, glycerol, pentaerythritol.

Component d) is preferably used in amounts of ≧0% and ≦10% by weight, more preferably ≧0% and ≦3% by weight, based on the total weight of the polyurethane urea, and is most preferably not used at all.

In addition, the polyurethane ureas may be formed from component e), one or more compounds having a group reactive toward isocyanate groups, especially compounds having an amino or hydroxyl group. Suitable compounds of component e) are, for example, methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine, methanol, ethanol, isopropanol, n-propanol, n-butanol, ethylene glycol monobutyl ether, diethylene glycol monomethyl ether, diethylene glycol monobutyl ether, propylene glycol monomethyl ether, dipropylene glycol monomethyl ether, tripropylene glycol monomethyl ether, dipropylene glycol monopropyl ether, propylene glycol monobutyl ether, dipropylene glycol monobutyl ether, tripropylene glycol monobutyl ether, 2-ethylhexanol, 1-octanol, 1-dodecanol, 1-hexadecanol.

Component e) preferably does not comprise any monofunctional polyether polyols having a proportion of groups obtained from ethylene oxide of >30% by weight, preferably >50% by weight.

The monohydroxy-functional alcohol used as solvent for the polyurethane urea can likewise serve as formation component e) for the polyurethane urea.

Component e) is used preferably in amounts of ≧0% and ≦10% by weight, more preferably ≧0% and ≦3% by weight, based on the total weight of the polyurethane urea, and is most preferably not used at all, not including the monohydroxy-functional alcohol used as solvent for the polyurethane urea as component e).

The monohydroxy-functional alcohol which serves as solvent for the polyurethane urea makes up preferably ≧0% and ≦5% by weight, more preferably ≧0.01% and ≦3% by weight and most preferably ≧0.01% and ≦2% by weight of the total mass of the polyurethane urea.

The polyurethane urea may also be formed from component f), a polyol or two or more polyols having a number average molecular weight Mₙ of ≧500 and ≦6000 g/mol and the hydroxyl functionality of ≧1.5 and ≦4, the polyols being different than b).

Component f) is preferably used in amounts of ≧0% and ≦20% by weight, more preferably ≧0% and ≦10% by weight, based on the total weight of the polyurethane urea, and is most preferably not used at all.

Preferably, the polyols of component f) have a number-average molecular weight Mₙ of ≧1000 and ≦3000 g/mol and a hydroxyl functionality of ≧1.8 and ≦3.

Polyols suitable as component f) are the following polyols that are known in polyurethane coating technology: polyester polyols, polyacrylate polyols, polyurethane polyols, polycarbonate polyols, polyester polyacrylate polyols, polyurethane polyacrylate polyols, polyurethane polyester polyols, polyurethane polyether polyols, polyurethane polycarbonate polyols, polyether polycarbonate polyols and/or polyester polycarbonate polyols, especially polyester polyols and/or polycarbonate polyols.

Polyester polyols are, for example, the polycondensates of di- and optionally tri- and tetraols and di- and optionally tri- and tetracarboxylic acids or hydroxycarboxylic acids or lactones. Instead of the free polycarboxylic acids, it is also possible to use the corresponding polycarboxylic anhydrides or corresponding polycarboxylic esters of lower alcohols to produce the polyesters.

Examples of diols suitable for this purpose are ethylene glycol, butylene glycol, diethylene glycol, triethylene glycol, polyalkylene glycols such as polyethylene glycol, and also propane-1,2-diol, propane-1,3-diol, butane-1,3-diol, butane-1,4-diol, hexane-1,6-diol and isomers, neopentyl glycol or neopentyl glycol hydroxypivalate, preference being given to hexane-1,6-diol and isomers, neopentyl glycol and neopentyl glycol hydroxypivalate. In addition, it is also possible to use polyols such as trimethylolpropane, glycerol, erythritol, pentaerythritol, trimethylolbenzene or trishydroxyethyl isocyanurate.

The dicarboxylic acids used may be phthalic acid, isophthalic acid, terephthalic acid, tetrahydrophthalic acid, hexahydrophthalic acid, cyclohexanedicarboxylic acid, adipic acid, azelaic acid, sebacic acid, glutaric acid, tetrachlorophthalic acid, maleic acid, fumaric acid, itaconic acid, malonic acid, suberic acid, 2-methylsuccinic acid, 3,3-diethylglutaric acid and/or 2,2-dimethylsuccinic acid. It is also possible to use the corresponding anhydrides as acid source.

If the mean hydroxyl functionality of the polyol to be esterified is greater than 2, it is additionally also possible to use monocarboxylic acids such as benzoic acid and hexanecarboxylic acid as well.

Preferred acids are aliphatic or aromatic acids of the aforementioned type. Particular preference is given to adipic acid, isophthalic acid and optionally trimellitic acid, very particular preference to adipic acid.

Examples of hydroxycarboxylic acids that may be used as reaction participants in the preparation of a polyester polyol having terminal hydroxyl groups include hydroxycaproic acid, hydroxybutyric acid, hydroxydecanoic acid, hydroxystearic acid and the like. Suitable lactones are caprolactone, butyrolactone and homologues. Preference is given to caprolactone.

In component f), it is also possible to use polycarbonates having hydroxyl groups, preferably polycarbonatediols, having number-average molecular weights Mₙ of 400 to 8000 g/mol, preferably of 600 to 3000 g/mol. These are obtainable by reaction of carbonic acid derivatives, such as diphenyl carbonate, dimethyl carbonate or phosgene, with polyols, preferably diols.

Examples of such diols are ethylene glycol, propane-1,2- and 1,3-diol, butane-1,3- and 1,4-diol, hexane-1,6-diol, octane-1,8-diol, neopentyl glycol, 1,4-bishydroxy-methylcyclohexane, 2-methylpropane-1,3-diol, 2,2,4-trimethylpentane-1,3-diol, dipropylene glycol, polypropylene glycols, dibutylene glycol, polybutylene glycols, bisphenol A, and lactone-modified diols of the aforementioned type. The polycarbonates having hydroxyl groups preferably have a linear structure.

In a preferred embodiment of the invention, the polyurethane urea used in accordance with the invention is formed from
a) at least one of an aliphatic, an araliphatic and a cycloaliphatic diisocyanate having at least one isocyanate group bonded to a secondary or tertiary carbon atom,
b) at least one polyether polyol having a number-average molecular weight Mₙ of ≧500 and ≦2500 g/mol and a hydroxyl functionality of ≧1.9 and ≦3,
c) at least one amino-functional compound having at least two (isocyanate-reactive) amino groups and selected from ethylenediamine, IPDA and/or H12-MDA,
d) optionally, at least one alcohol having at least two hydroxyl groups and a molar mass of ≧60 and ≦399 g/mol,
e) optionally, at least one compound having a group reactive toward isocyanate groups and
f) optionally, ≦20% by weight, based on the total mass of the polyurethane urea, of at least one different polyol than b) having a number-average molecular weight Mₙ of ≧500 and ≦6000 g/mol and a hydroxyl functionality of ≧1.5 and ≦4.

Further preferably, the polyurethane urea, in this aforementioned embodiment, is formed from ≧5% and ≦60% by weight of component a), ≧30% and ≦90% by weight of component b), ≧2% and ≦25% by weight of component c), ≧0% and ≦10% by weight of component d), ≧0% and ≦10% by weight of component e) and ≧0% and ≦20% by weight of component f), based in each case on the total mass of the polyurethane urea, where components a) to f) add up to 100% by weight.

Especially preferably, the polyurethane urea, in this aforementioned embodiment, is formed from ≧10% and ≦40% by weight of component a), ≧55% and ≦85% by weight of component b), ≧5% and ≦20% by weight of component c), ≧0% and ≦3% by weight of component d), ≧0% and ≦3% by weight of component e) and ≧0% and ≦1% by weight of component f), based in each case on the total mass of the polyurethane urea, where components a) to f) add up to 100% by weight.

In a particularly preferred embodiment of the invention, the polyurethane urea used in accordance with the invention is formed from
a) at least one isocyanate selected from IPDI and H12-MDI,
b) at least one polyether polyol having a number average molecular weight Mₙ≧500 and ≦2500 and a hydroxyl functionality of ≧1.9 and ≦3, selected from polypropylene glycols and/or poly(tetramethylene glycol) polyether polyols,
c) at least one amino-functional compound selected from IPDA and H12-MDA,
d) optionally, at least one alcohol having at least two hydroxyl groups and a molar mass of ≧60 and ≦399 g/mol,
e) optionally, at least one compound having a group reactive toward isocyanate groups and
f) optionally, ≦20% by weight, based on the total mass of the polyurethane urea, of at least one different polyol than b) having a number-average molecular weight Mₙ of ≧500 and ≦6000 g/mol and a hydroxyl functionality of ≧1.5 and ≦4.

Further preferably, the polyurethane urea, in this aforementioned embodiment, is formed from ≧5% and ≦60% by weight of component a), ≧30% and ≦90% by weight of component b), ≧2% and ≦25% by weight of component c), ≧0% and ≦10% by weight of component d), ≧0% and ≦10% by weight of component e) and ≧0% and ≦20% by weight of component f), based in each case on the total mass of the polyurethane urea, where components a) to f) add up to 100% by weight.

Especially preferably, the polyurethane urea, in this aforementioned embodiment, is formed from ≧10% and ≦40% by weight of component a), ≧55% and ≦85% by weight of component b), ≧5% and ≦20% by weight of component c), ≧0% and ≦3% by weight of component d), ≧0% and ≦3% by weight of component e) and ≧0% and ≦1% by weight of component f), based in each case on the total mass of the polyurethane urea, where components a) to f) add up to 100% by weight.

Preferably, the polyurethane urea is formed from components a) to c) and optionally d) to f), more preferably from components a) to c).

Advantageously, the polyurethane urea has a number-average molecular weight Mₙ≧2000 and ≦50 000 g/mol, particularly advantageously ≧3000 and ≦30 000 g/mol.

The polyurethane urea is preferably prepared by reacting components a) and b) and optionally d) and f) in a first step to give an NCO-terminated prepolymer, which is then reacted in a subsequent step with component c) and optionally components d) and e).

For the preparation of the polyurethane ureas, preferably, components a) and b) and optionally d) and f) for preparation of an NCO-terminated prepolymer are initially charged in full or in part, optionally diluted with a solvent inert toward isocyanate groups, and heated up to temperatures in the range from 50 to 120° C. The isocyanate addition reaction can be accelerated using the catalysts known in polyurethane chemistry. A preferred variant, however, works without the addition of urethanization catalysts.

Subsequently, any constituents of a) and b) and optionally d) and f) which have not yet been added at the start of the reaction are metered in.

In the preparation of the NCO-terminated prepolymers from components a) and b) and optionally d) and f), the molar ratio of isocyanate groups to isocyanate reactive groups is generally ≧1.05 and ≦3.5, preferably ≧1.1 and ≦3.0, more preferably ≧1.1 and ≦2.5.

Isocyanate-reactive groups are understood to mean all groups reactive toward isocyanate groups, for example primary and secondary amino groups, hydroxyl groups or thiol groups.

The conversion of components a) and b) and optionally d) and f) to the prepolymer is effected in part or in full, but preferably in full. In this way, polyurethane prepolymers containing free isocyanate groups are obtained in substance or in solution.

Preferably, the NCO-terminated prepolymer is prepared from components a) and b).

Thereafter, preferably, in a further process step, if this has been done only partly, if at all, the prepolymer obtained is dissolved with the aid of one or more organic solvents. The solvent used is preferably likewise a solvent or solvent mixture, where the solvent consists of one or more monohydroxy-functional alcohols or a solvent mixture consisting of organic solvents containing ≧50% by weight, based on the total mass of the solvent mixture, of at least one monohydroxy-functional alcohol is used. In respect of the solvent and solvent mixture, the preferred embodiments below relating to the solvent or solvent mixture in which the polyurethane urea is dissolved are likewise applicable. The solvent or solvent mixture may also be different than the solvent or solvent mixture in which the polyurethane urea as end product is dissolved at a later stage. The solvent or solvent mixture is preferably identical to the solvent or solvent mixture in which the polyurethane urea as end product is dissolved at a later stage.

Preferably, the solvent used in the preparation consists of one or more monohydroxy-functionalized alcohols.

The ratio of solvent to prepolymer is preferably ≧1:10 and ≦5:1, more preferably ≧1:2 and ≦2:1, parts by weight.

Prior to the dissolution, the prepolymer is cooled down to temperatures of −20 to 60° C., preferably 0 to 50° C. and more preferably 15 to 40° C.

In a further step that optionally follows the dissolution of the NCO-terminated prepolymer, the NCO-terminated prepolymer obtained in the first step is then preferably reacted fully or partly with component c) and optionally components d) and e). This reaction is generally referred to as chain extension, or in the case of component e) as chain termination.

Preference is given here to initially charging the NCO-terminated prepolymer, and metering in components c) and optionally d) and e). Preference is given to firstly partly reacting the NCO groups of the prepolymer with components c) and optionally d), followed by chain termination by reaction of the remaining NCO groups with component e). Components c) and optionally e) may also be added stepwise in two or more steps, especially in two steps.

Component c) and optionally d) and e) are preferably used dissolved in one or more organic solvents. The solvent used is preferably likewise a solvent or solvent mixture, where the solvent consists of one or more monohydroxy-functional alcohols or a solvent mixture consisting of organic solvents containing ≧50% by weight, based on the total mass of the solvent mixture, of at least one monohydroxy-functional alcohol is used. In respect of the solvent and solvent mixture, the preferred embodiments below relating to the solvent or solvent mixture in which the polyurethane urea is dissolved are likewise applicable.

The solvent or solvent mixture may also be different than the solvent or solvent mixture in which the polyurethane urea as end product is dissolved at a later stage. The solvent or solvent mixture is preferably identical to the solvent or solvent mixture in which the polyurethane urea as end product is dissolved at a later stage.

Preferably, the solvent used in the preparation for component c) consists of one or more monohydroxy-functionalized alcohols.

When solvents are used as diluents, the diluent content in the components c) used in the chain extension, and optionally d) and e), is preferably 1% to 95% by weight, preferably 3% to 50% by weight, based on the total weight of component c) and optionally d) and e) including diluents.

Components c) and optionally d) and e) are preferably added at temperatures of −20 to 60° C., preferably 0 to 50° C. and more preferably of 15 to 40° C.

The degree of chain extension, i.e. the molar ratio of NCO-reactive groups of the components c) used for chain extension and chain termination, and optionally d) and e), to free NCO groups of the prepolymer, is ≧50 and ≦150%, preferably ≧50 and ≦120%, more preferably ≧60 and ≦100% and most preferably ≧70 and ≦95%.

Preferably, the molar ratio of isocyanate-reactive groups of component c) to the free NCO groups of the prepolymer is ≧50% and ≦120%, more preferably ≧60% and ≦100% and most preferably ≧70% and ≦95%.

In a preferred embodiment of the invention, the free NCO groups of the prepolymer are only partly reacted with component c), the molar ratio of isocyanate-reactive groups of component c) to the free NCO groups of the prepolymer preferably being ≧60% and ≦95% and the remaining free NCO groups being depleted by reaction with the hydroxyl groups of the solvent, so as to form an NCO-free polyurethane urea.

After the preparation, the polyurethane urea, if solvents or solvent mixtures of the invention have already been used in the preparation process, can still be diluted and dissolved with a solvent or solvent mixture, in which case the solvent consists of one or more monohydroxy-functional alcohols or a solvent mixture consisting of organic solvents containing ≧50% by weight, based on the total mass of the solvent mixture, of at least one monohydroxy-functional alcohol is used.

If no solvents or solvent mixtures have been used during the reaction, after the polyurethane urea has been prepared, it is used in a solvent or solvent mixture, in which case the solvent consists of one or more monohydroxy-functional alcohols or a solvent mixture consisting of organic solvents and containing ≧50% by weight, based on the total mass of the solvent mixture, of at least one monohydroxy-functional alcohol is used.

The dissolution of the polyurethane urea can be effected by standard techniques for shearing, for example by stirring with standard stirrers as specified in DIN 28131. The polyurethane urea is preferably dissolved without the additional addition of external emulsifiers. The polyurethane urea solutions used in accordance with the invention preferably do not comprise any external emulsifiers. Suitable solvents or constituents of the solvent mixture are in principle all monohydroxy-functional aliphatic alcohols having one to six carbon atoms, for example methanol, ethanol, n-propanol, isopropanol, n-butanol, sec-butanol and/or butylglycol. More preferably, the monohydroxy-functional alcohol is ethanol.

If a solvent mixture is used, as well as the monohydroxy-functional alcohols, it is also possible to use ≦50% by weight, based on the total mass of the solvent mixture, of a further organic solvent. Suitable solvents here are, for example, esters, for example ethyl acetate, butyl acetate, methoxypropyl acetate or butyrolactone, ketones, for example acetone or methyl ethyl ketone, ethers, for example tetrahydrofuran or tert-butyl methyl ether, aromatic solvents, for example xylene or solvent naphtha. In the case of use of ethanol, typical denaturing agents may be present as additives in the customary added amounts.

Preferably, the proportion of the further organic solvents is ≦30% by weight, more preferably ≦5% by weight and most preferably ≦2% by weight, based on the total weight of the solvent mixture. In a most preferred embodiment, no further organic solvents are present aside from monohydroxy-functional aliphatic alcohols.

Unsuitable further solvents are physiologically incompatible solvents, for example dimethylformamide, N-methylpyrrolidone or toluene, as often used as co-solvents for polyurethanes or polyurethane ureas, thus these should preferably not be present in cosmetic compositions.

The further solvents are not water. The polyurethane urea solution obtained by dissolving the polyurethane urea in the solvents or solvent mixtures used in accordance with the invention is preferably anhydrous, excluding the proportions of water present as a result of the preparation in the organic solvents used.

The water content of the polyurethane urea solution is ≦10% by weight, preferably ≦4.5% by weight and most preferably ≦1% by weight, based on the total mass of the polyurethane urea solution.

The proportion of the polyurethane urea (as active substance) in the polyurethane urea solution used in accordance with the invention (also referred to as solids content) is preferably ≧10% and ≦80% by weight, more preferably ≧15% and ≦60% by weight and most preferably ≧20% and ≦50% by weight, based on the total weight of the polyurethane urea solution.

The process of the invention is suitable for production of cosmetic compositions, or the cosmetic compositions obtainable by the process of the invention are preferably those that are employed for treatment of nails, the skin and/or keratinic fibers, preferably of skin and/or hair, more preferably the skin, and they are especially sunscreen compositions. More preferably, they are sunscreen compositions for application to the skin.

Cosmetic compositions for treatment of nails are especially understood to mean nail varnishes.

Nails in the context of this invention are understood to mean fingernails and/or toenails.

The invention further provides a cosmetic composition obtainable by the process of the invention.

The process of the invention is suitable for production of cosmetic compositions, or the cosmetic compositions obtainable by the process of the invention are preferably those that are in the form of gels, oils, sprays and aerosols that are preferably transparent. “Transparent” in the context of the present invention means that the turbidity values of the composition are ≦100 NTU (Nephelometric Turbidity Unit), preferably ≦50 NTU, more preferably ≦10 NTU and most preferably ≦5 NTU. Turbidity values are determined by a scattered light measurement at a 90° angle (nephelometry) at a measurement radiation wavelength of 860 nm in accordance with DIN EN ISO 7027, conducted at 23° C. with a model 2100AN laboratory turbidimeter from HACH LANGE GmbH, Berlin, Germany.

Preferably, the cosmetic compositions are those that are predominantly alcohol-based, i.e. contain ≧10% and ≦90% by weight, based on the total mass of the cosmetic composition, preferably ≧15% and ≦70% by weight and more preferably ≧20% and ≦60% by weight of aliphatic alcohols having 1 to 6 carbon atoms, preferably 1 to 4 carbon atoms. The alcohols are preferably selected from ethanol and isopropanol; polyol and derivatives thereof, such as propylene glycol, dipropylene glycol, butylene 1,3-glycol, polypropylene glycol, glycol ethers such as alkyl (C1-4) ethers of mono-, di- or tripropylene glycol or mono-, di- or triethylene glycol, or mixtures thereof. More preferably, the alcohols contain ethanol or consist thereof; most preferably, the alcohol used is ethanol.

More preferably, the cosmetic compositions are alcoholic solutions.

The cosmetic compositions preferably contain a water content of ≧0% and ≦30% by weight, more preferably ≧0% and ≦20% by weight, even more preferably of ≧0% and ≦5% by weight and further preferably of ≧0% and <2% by weight. Especially preferably, the cosmetic compositions are anhydrous, and thus contain no more water than what is unavoidably introduced into the formulation via the raw materials as a result of production.

The proportion of the polyurethane urea solution used in the cosmetic composition is preferably ≧0.5% and ≦80% by weight, more preferably ≧1% and ≦60% by weight and most preferably ≧2% and ≦40% by weight, based on the total mass of the cosmetic composition.

The solids content of the polyurethane urea solution is preferably chosen such that the cosmetic compositions contain preferably ≧0.1% and ≦30% by weight, more preferably ≧0.5% and ≦20% by weight and most preferably ≧1% and ≦10% by weight of the polyurethane urea as active substance, based on the total mass of the cosmetic composition.

Active substance is understood to mean the polyurethane urea without solvent or solvent mixture.

The cosmetic compositions additionally preferably contain additives customary in cosmetics, such as emulsifiers, interface-active substances, defoamers, thickeners, surfactants, humectants, filler, film former, solvent, coalescent, gel former and/or other polymer dispersions, for example dispersions based on polyacrylates, fillers, plasticizers, pigments, dyes, leveling agents, thixotropic agents, sleekness agents, preservatives, sensory additives, oils, waxes and/or propellant gases, for example, propane/butane or dimethyl ether, etc. The amounts of the various additives are known to the person skilled in the art for the range to be used and are, for example, in the range of ≧0% and ≦40% by weight, preferably ≧0.1% and ≦40% by weight, based on the total weight of the cosmetic composition.

Preferably, the cosmetic compositions also comprise sunscreen filter substances, especially UV absorbers. The proportion of the sunscreen filter substances in the total mass of the cosmetic composition is preferably ≧0.01% and ≦40% by weight, more preferably ≧1% and ≦35% by weight and most preferably ≧5% and ≦30% by weight.

The terms “sunscreen filter substances” and “UV filter substances” are used as equivalent terms in the context of this application.

The sunscreen filter substances (or UV filters) may be selected from the organic filters, the physical filters and/or mixtures thereof, but preference is given to organic filters, especially preferably oil-soluble organic filters. Suitable sunscreen filter substances are all those listed in Annex VII of the EU Cosmetics Directive (76/768/EEC).

The UV filters used may be oil-soluble and/or water-soluble, preference being given to oil-soluble UV filters. Preferably, the cosmetic composition comprises at least one oil-soluble UV filter.

Oil-soluble UV filters may be those that are liquid, especially oil-like, and may themselves also serve as solvents for other oil-soluble UV filters or those that are solid and are used dissolved in oils.

Liquid oil-soluble UV filters used with preference are octocrylene, ethylhexyl methoxycinnamate, ethylhexyl salicylate and/or homosalate.

Solid oil-soluble UV filters used with preference are butylmethoxydibenzoylmethane (Avobenzone), dioctylbutylamidotriazone (INCI: diethylhexyl butamidotriazone), 2,4-bis{[4-(2-ethylhexyloxy)-2-hydroxy]phenyl}-6-(4-methoxyphenyl)-1,3,5-triazine (INCI: bis-ethylhexyloxyphenol methoxyphenyl triazine), ethylhexyl triazone, diethylamino hydroxybenzoyl hexyl benzoate, benzophenone-3. In addition, it is also possible to use all other oil-soluble filters listed in Annex VII of the EU Cosmetics Directive (76/768/EEC).

In a preferred embodiment of the invention, the cosmetic compositions comprise at least one liquid oil-soluble UV filter.

The cosmetic compositions containing UV filters also include those cosmetic compositions whose main purpose is not protection from sunlight but which nevertheless contain a content of UV filters. For example, UV-A or UV-B filter substances are usually incorporated into day creams or makeup products. Haircare product or nail varnishes may also contain UV filter substances. In addition, UV protection substances, just like antioxidants and preservatives, constitute effective protection of the formulations themselves from spoilage.

The cosmetic formulations may contain oils and/or waxes, where the oils may be non-volatile and/or volatile oils.

The cosmetic composition advantageously contains ≧0% and ≦45% by weight of oils, based on the total weight of the composition, and particularly advantageously ≧0.01% and ≦20% by weight of oils.

The waxes may be present in amounts of ≧0% and ≦10% by weight, based on the total weight of the composition, and preferably ≧1% and ≦5% by weight.

In a preferred embodiment of the invention, the cosmetic composition comprises
A) at least one polyurethane urea dissolved in a solvent or solvent mixture, where the solvent consists of one or more monohydroxy-functional alcohols or a solvent mixture consisting of organic solvents containing ≧50% by weight, based on the total mass of the solvent mixture, of at least one monohydroxy-functional alcohol is used,
B) at least one oil-soluble sunscreen filter substance and
C) at least one aliphatic alcohol having 1 to 6 carbon atoms.

In a particularly preferred embodiment of the invention, the cosmetic composition comprises
A) ≧0.5% and ≦80% by weight of at least one polyurethane urea dissolved in a solvent or solvent mixture, where the solvent consists of one or more monohydroxy-functional alcohols or a solvent mixture consisting of organic solvents containing ≧50% by weight, based on the total mass of the solvent mixture, of at least one monohydroxy-functional alcohol is used,
B) ≧0.01% and ≦40% by weight of sunscreen filter substances, comprising at least one oil-soluble sunscreen filter substance,
C) ≧10% and ≦90% by weight of at least one aliphatic alcohol having 1 to 4 carbon atoms,
D) ≧0% and ≦45% by weight of at least one oil and/or ≧0% and ≦10% by weight of at least one wax, and
E) ≧0% and ≦40% by weight of additives customary in cosmetics,
   based in each case on the total mass of the cosmetic composition, where components A) to D) add up to 100% by weight.

The content of component D) is preferably =0 only when component B) already contains at least one liquid oil-soluble sunscreen filter substance. The latter can then be regarded as an oil which is also able to dissolve further solid oil-soluble sunscreen filter substances.

The solids content of the polyurethane urea solution is preferably chosen such that the cosmetic compositions contain preferably ≧0.5% and ≦20% by weight of the polyurethane urea as active substance, based on the total mass of the cosmetic composition.

The process of the invention preferably comprises a step in which a homogeneous oil phase is produced from the oil-soluble UV filter substances together with any further oils and waxes, and optionally further additives. This step is preferably conducted at elevated temperatures, more preferably ≧20° C. and ≦90° C. If the oil phase is produced at temperatures above room temperature, it can be cooled after the production, preferably to room temperature.

In addition, the process of the invention preferably comprises a step in which the oil phase is mixed with a second phase comprising at least the polyurethane urea solution. This further phase preferably also comprises at least one aliphatic alcohol having 1 to 6 and preferably 1 to 4 carbon atoms.

Additives can be introduced into the cosmetic composition at any time in the process.

The invention further provides a process for producing a cosmetic coating on skin, nails and/or keratinic fibers, preferably on skin and/or hair, more preferably on the skin, using the cosmetic compositions of the invention, wherein the cosmetic composition is applied to skin, nails and/or keratinic fibers, preferably to skin and/or hair, more preferably to the skin.

Advantageously, in the process of the invention, the cosmetic compositions of the invention remain at least partly on the skin, nails and/or keratinic fibers, preferably on skin and/or hair, more preferably on the skin.

The invention further provides for the use of the cosmetic composition of the invention for cosmetic coating of nails, skin and/or keratinic fibers, preferably of skin and/or hair, more preferably the skin.

The invention further provides a sunscreen composition comprising at least one sunscreen filter substance and at least one polyurethane urea which has no ionically hydrophilizing groups and which is used dissolved in a solvent or solvent mixture, wherein the solvent consists of one or more monohydroxy-functional alcohols or a solvent mixture consisting of organic solvents and containing ≧50% by weight, based on the total mass of the solvent mixture, of at least one monohydroxy-functional alcohol is used. The polyurethane urea used is formed from
a) at least one of an aliphatic, an araliphatic and a cycloaliphatic diisocyanate,
b) at least one polyether polyol having a number-average molecular weight Mₙ of ≧400 and ≦6000 g/mol and a hydroxyl functionality of ≧1.5 and ≦4,
c) at least one amino-functional compound having at least two isocyanate-reactive amino groups,
d) optionally, at least one alcohol having at least two hydroxyl groups and a molar mass of ≧60 and ≦399 g/mol,
e) optionally, at least one compound having a group reactive toward isocyanate groups and
f) optionally, ≦20% by weight, based on the total mass of the polyurethane urea, of at least one different polyol than b) having a number-average molecular weight Mₙ of ≧500 and ≦6000 g/mol and a hydroxyl functionality of ≧1.5 and ≦4.

The polyurethane ureas of the present invention are used for production of the sunscreen composition of the invention in dissolved form in a solvent or solvent mixture, and hence as polyurethane urea solutions and not as aqueous dispersions.

Ionically hydrophilizing groups in the context of the invention are those which can be introduced into the polyurethane urea, for example, by means of suitable anionically or potentially anionically hydrophilizing compounds. These have at least one isocyanate-reactive group, such as a hydroxyl or amino group, and at least one functionality, for example, —COO-M⁺, —SO₃-M⁺, —PO(O-M⁺)₂ where M⁺, for example is a metal cation, H⁺, NH₄⁺, NHR₃⁺ where each R is a C₁-C₁₂-alkyl radical, C₅-C₆-cycloalkyl radical and a C₂-C₄-hydroxyalkyl radical, which enters into a pH-dependent dissociation equilibrium on interaction with aqueous media and in this way may be negatively charged or uncharged. Suitable anionically or potentially anionically hydrophilizing compounds are mono- and dihydroxycarboxylic acids, mono- and dihydroxysulfonic acids, and mono- and dihydroxyphosphonic acids and salts thereof. Examples of such anionic or potentially anionic hydrophilizing agents are dimethylolpropionic acid, dimethylolbutyric acid, hydroxypivalic acid, malic acid, citric acid, glycolic acid, lactic acid and the propoxylated adduct of 2-butenediol and NaHSO₃, as described in DE-A 2 446 440, pages 5-9, formula I-III.

Potentially anionic (and also generally potentially ionic) groups can be converted, especially by neutralization, to an anionic (ionic) group in the chemical reaction.

In a preferred embodiment of the process of the invention, the polyurethane urea used does not have any hydrophilizing groups, i.e. neither ionically nor nonionically hydrophilizing groups.

Nonionic hydrophilizing groups in the context of the invention are those which could be introduced into the polyurethane urea, for example, by means of suitable nonionically hydrophilizing compounds, for example polyoxyalkylene ethers containing at least one hydroxyl or amino group. Examples are the monohydroxy-functional polyalkylene oxide polyether alcohols having a statistical average of 5 to 70, preferably 7 to 55, ethylene oxide units per molecule, as obtainable in a manner known per se by alkoxylation of suitable starter molecules (described, for example, in Ullmanns Encyclopadie der technischen Chemie [Ullmann's Encyclopedia of Industrial Chemistry], 4th edition, volume 19, Verlag Chemie, Weinheim p. 31-38). These compounds are either pure polyethylene oxide ethers or mixed polyalkylene oxide ethers, in which case, however, they contain at least 30 mol %, preferably at least 40 mol %, based on all alkylene oxide units present, of ethylene oxide units.

The polyurethane urea used in accordance with the invention formed from
a) at least one of an aliphatic, an araliphatic and a cycloaliphatic diisocyanate,
b) at least one polyether polyol having a number-average molecular weight Mₙ of ≧400 and ≦6000 g/mol and a hydroxyl functionality of ≧1.5 and ≦4,
c) at least one amino-functional compound having at least two isocyanate-reactive amino groups,
d) optionally, at least one alcohol having at least two hydroxyl groups and a molar mass of ≧60 and ≦399 g/mol,
e) optionally, at least one compound having a group reactive toward isocyanate groups and
f) optionally, ≦20% by weight, based on the total mass of the polyurethane urea, of at least one different polyol than b) having a number-average molecular weight Mₙ of ≧500 and ≦6000 g/mol and a hydroxyl functionality of ≧1.5 and ≦4.

Further preferably, the polyurethane urea is formed from ≧5% and ≦60% by weight of component a), ≧30% and ≦90% by weight of component b), ≧2% and ≦25% by weight of component c), ≧0% and ≦10% by weight of component d), ≧0% and ≦10% by weight of component e) and ≧0% and ≦20% by weight of component f), based in each case on the total mass of the polyurethane urea, where components a) to f) add up to 100% by weight.

Especially preferably, the polyurethane ureas is formed from ≧10% and ≦40% by weight of component a), ≧55% and ≦85% by weight of component b), ≧5% and ≦20% by weight of component c), ≧0% and ≦3% by weight of component d), ≧0% and ≦3% by weight of component e) and ≧0% and ≦1% by weight of component f), based in each case on the total mass of the polyurethane urea, where components a) to f) add up to 100% by weight.

Compounds suitable as component a) are, for example, butylene 1,4-diisocyanate, pentamethylene 1,5-diisocyanate (PDI), hexamethylene 1,6-diisocyanate (HDI), isophorone diisocyanate (IPDI), 2,2,4- and/or 2,4,4-trimethylhexamethylene diisocyanate, the isomeric bis(4,4′-isocyanatocyclohexyl)methanes or mixtures thereof with any isomer content (H12-MDI), cyclohexylene 1,4-diisocyanate, 4-isocyanatomethyloctane 1,8-diisocyanate (nonane triisocyanate), 1,3- and/or 1,4-bis(2-isocyanatoprop-2-yl)benzene (TMXDI), 1,3-bis(isocyanatomethyl)benzene (XDI) and alkyl 2,6-diisocyanatohexanoates (lysine diisocyanates) having C₁-C₈-alkyl groups.

As well as the aforementioned polyisocyanates, it is also possible to use proportions of modified diisocyanates or triisocyanates having isocyanurate, urethane, allophanate, biuret, iminooxadiazinedione and/or oxadiazinetrione structure.

Preferably, the polyisocyanates or polyisocyanate mixtures are of the aforementioned type with a mean NCO functionality of ≧2 and ≦4, preferably of ≧2 and ≦2.6 and more preferably of ≧2 and ≦2.4.

Preferably, component a) is selected from aliphatic, araliphatic and/or cycloaliphatic diisocyanates having at least one isocyanate group bonded to a secondary and/or tertiary carbon atom.

More preferably, component a) is selected from IPDI and/or H12-MDI.

Further preferably, no aromatic polyisocyanates are used for preparation of the polyurethane urea.

Component a) is preferably used in amounts of ≧5% and ≦60% by weight, more preferably ≧10% and ≦40% by weight and most preferably of ≧15% and ≦35% by weight, based on the total weight of the polyurethane ureas.

Component b) consists of one or more polyether polyols having a number-average molecular weight Mₙ≧400 and ≦6000 g/mol and a hydroxyl functionality of ≧1.5 and ≦4, preferably having a number-average molecular weight Mₙ≧500 and ≦2500 g/mol and a hydroxyl functionality of ≧1.9 and ≦3 and more preferably having a number-average molecular weight Mₙ≧1000 and ≦2000 g/mol and a hydroxyl functionality of ≧1.9 and ≦2.1.

Suitable polyether polyols of component b) are, for example, the poly(tetramethylene glycol) polyether polyols known in polyurethane chemistry, as obtainable by polymerization of tetrahydrofuran by means of cationic ring opening.

Likewise suitable polyether polyols are the addition products of styrene oxide, ethylene oxide, propylene oxide, butylene oxide and/or epichlorohydrin onto di- or polyfunctional starter molecules. Polyalkylene glycols in particular, such as polyethylene glycols, polypropylene glycols and/or polybutylene glycols, are applicable, especially with the abovementioned preferred molecular weights. The polyether polyols preferably have a proportion of groups obtained from ethylene oxide of <50% by weight, preferably <30% by weight.

Suitable starter molecules used may be all compounds known according to prior art, for example water, butyldiglycol, glycerol, diethylene glycol, trimethylolpropane, propylene glycol, sorbitol, ethylenediamine, triethanolamine, butane-1,4-diol.

Preferably, component b) are selected from polypropylene glycols and/or poly(tetramethylene glycol) polyether polyols, more preferably selected from poly(tetramethylene glycol) polyether polyols.

In a preferred employment of the invention, component b) comprises one or more poly(tetramethylene glycol) polyether polyols having a number-average molecular weight Mₙ≧500 and ≦2500 g/mol and a hydroxyl functionality of ≧1.9 and ≦2.1.

In a particularly preferred embodiment, component b) is a mixture of poly(tetramethylene glycol) polyether polyols I having a number-average molecular weight Mₙ of ≧400 and ≦1500 g/mol, more preferably of ≧600 and ≦1200 g/mol, most preferably of 1000 g/mol, and poly(tetramethylene glycol) polyether polyols II having a number-average molecular weight Mₙ of ≧1500 and ≦8000 g/mol, more preferably of ≧1800 and ≦3000 g/mol, most preferably of 2000 g/mol.

The ratio of poly(tetramethylene glycol) polyether polyols I to the poly(tetramethylene glycol) polyether polyols II is preferably in the range from ≧0.1 to ≦10, more preferably in the range from ≧0.2 to ≦8 and most preferably in the range from ≧1 to ≦6.

Component b) is preferably used in amounts of ≧30% and ≦90% by weight, more preferably ≧50% and ≦85% by weight, most preferably of ≧55% and ≦75% by weight, based on the total weight of the polyurethane urea.

Component c) is one or more amino-functional compounds having at least two isocyanate-reactive groups.

Suitable components c) are, for example, di- or polyamines such as ethylene-1,2-diamine, 1,2- and 1,3-diaminopropane, 1,4-diaminobutane, 1,6-diaminohexane, 2,2,4- and 2,4,4-trimethylhexamethylenediamine, 2-methylpentamethylenediamine, diethylenetriamine, triaminononane, xylylene-1,3- and 1,4-diamine, α,α,α′,α′-tetramethylxylylene-1,3- and -1,4-diamine and 4,4′-diaminodicyclohexylmethane (H12-MDA), isophoronediamine (IPDA) and/or 1,2-dimethylethylenediamine.

Preferably, component c) is selected from ethyleneamine, IPDA and/or H12-MDA, more preferably from isophoronediamine and/or H12-MDA, and component c) is most preferably H12-MDA.

The compounds of component c) preferably do not contain any hydrophilizing groups, and more particularly no ionically or potentially anionically hydrophilizing groups.

In a particularly preferred embodiment of the invention, component c) is selected from amines having at least two isocyanate-reactive amino groups bonded to primary and/or secondary carbon atoms.

Further preferably, component c) is selected from diamines of symmetric structure. Most preferably, component c) is selected from symmetric diamines having at least two amino groups bonded to primary and/or secondary carbon atoms; component c) is especially preferably H12-MDA.

Component c) is preferably used in amounts of ≧2% and ≦25% by weight, more preferably ≧5% and ≦20% by weight and most preferably ≧9% and ≦16% by weight, based on the total weight of the polyurethane urea.

In a preferred embodiment of the invention, either component a) is H12-MDI or component c) is H12-MDA or component a) is H12-MDI and component c) is H12-MDA.

Optionally, the polyurethane urea is additionally formed from component d), one or more alcohols having at least two hydroxyl groups and a molar mass of ≧60 and ≦399 g/mol, for example polyols of the molar mass range mentioned having up to 20 carbon atoms, such as ethylene glycol, diethylene glycol, triethylene glycol, propane-1,2-diol, propane-1,3-diol, butane-1,4-diol, 1,3-butylene glycol, cyclohexanediol, cyclohexane-1,4-dimethanol, hexane-1,6-diol, neopentyl glycol, hydroquinone dihydroxyethyl ether, bisphenol A (2,2-bis(4-hydroxyphenyl)propane), hydrogenated bisphenol A (2,2-bis(4-hydroxyphenyl)propane), trimethylolpropane, glycerol, pentaerythritol.

Component d) is preferably used in amounts of ≧0% and ≦10% by weight, more preferably ≧0% and ≦3% by weight, based on the total weight of the polyurethane urea, and is most preferably not used at all.

In addition, the polyurethane ureas may be formed from component e), one or more compounds having a group reactive toward isocyanate groups, especially compounds having an amino or hydroxyl group. Suitable compounds of component e) are, for example, methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine, methanol, ethanol, isopropanol, n-propanol, n-butanol, ethylene glycol monobutyl ether, diethylene glycol monomethyl ether, diethylene glycol monobutyl ether, propylene glycol monomethyl ether, dipropylene glycol monomethyl ether, tripropylene glycol monomethyl ether, dipropylene glycol monopropyl ether, propylene glycol monobutyl ether, dipropylene glycol monobutyl ether, tripropylene glycol monobutyl ether, 2-ethylhexanol, 1-octanol, 1-dodecanol, 1-hexadecanol.

Component e) preferably does not comprise any monofunctional polyether polyols having a proportion of groups obtained from ethylene oxide of >30% by weight, preferably >50% by weight.

The monohydroxy-functional alcohol used as solvent for the polyurethane urea can likewise serve as formation component e) for the polyurethane urea.

Component e) is used preferably in amounts of ≧0% and ≦10% by weight, more preferably ≧0% and ≦3% by weight, based on the total weight of the polyurethane urea, and is most preferably not used at all, not including the monohydroxy-functional alcohol used as solvent for the polyurethane urea as component e).

The monohydroxy-functional alcohol which serves as solvent for the polyurethane urea makes up preferably ≧0% and ≦5% by weight, more preferably ≧0.01% and ≦3% by weight and most preferably ≧0.01% and ≦2% by weight of the total mass of the polyurethane urea.

The polyurethane urea may also be formed from component f), a polyol or two or more polyols having a number average molecular weight Mₙ of ≧500 and ≦6000 g/mol and the hydroxyl functionality of ≧1.5 and ≦4, the polyols being different than b).

Component f) is preferably used in amounts of ≧0% and ≦20% by weight, more preferably ≧0% and ≦10% by weight, based on the total weight of the polyurethane urea, and is most preferably not used at all.

Preferably, the polyols of component f) have a number-average molecular weight Mₙ of ≧1000 and ≦3000 g/mol and a hydroxyl functionality of ≧1.8 and ≦3.

Polyols suitable as component f) are the following polyols that are known per se in polyurethane coating technology: polyester polyols, polyacrylate polyols, polyurethane polyols, polycarbonate polyols, polyester polyacrylate polyols, polyurethane polyacrylate polyols, polyurethane polyester polyols, polyurethane polyether polyols, polyurethane polycarbonate polyols, polyether polycarbonate polyols and/or polyester polycarbonate polyols, especially polyester polyols and/or polycarbonate polyols.

Polyester polyols are, for example, the polycondensates of di- and optionally tri- and tetraols and di- and optionally tri- and tetracarboxylic acids or hydroxycarboxylic acids or lactones. Instead of the free polycarboxylic acids, it is also possible to use the corresponding polycarboxylic anhydrides or corresponding polycarboxylic esters of lower alcohols to produce the polyesters.

Examples of diols suitable for this purpose are ethylene glycol, butylene glycol, diethylene glycol, triethylene glycol, polyalkylene glycols such as polyethylene glycol, and also propane-1,2-diol, propane-1,3-diol, butane-1,3-diol, butane-1,4-diol, hexane-1,6-diol and isomers, neopentyl glycol or neopentyl glycol hydroxypivalate, preference being given to hexane-1,6-diol and isomers, neopentyl glycol and neopentyl glycol hydroxypivalate. In addition, it is also possible to use polyols such as trimethylolpropane, glycerol, erythritol, pentaerythritol, trimethylolbenzene or trishydroxyethyl isocyanurate.

The dicarboxylic acids used may be phthalic acid, isophthalic acid, terephthalic acid, tetrahydrophthalic acid, hexahydrophthalic acid, cyclohexanedicarboxylic acid, adipic acid, azelaic acid, sebacic acid, glutaric acid, tetrachlorophthalic acid, maleic acid, fumaric acid, itaconic acid, malonic acid, suberic acid, 2-methylsuccinic acid, 3,3-diethylglutaric acid and/or 2,2-dimethylsuccinic acid. It is also possible to use the corresponding anhydrides as acid source.

If the mean hydroxyl functionality of the polyol to be esterified is greater than 2, it is additionally also possible to use monocarboxylic acids such as benzoic acid and hexanecarboxylic acid as well.

Preferred acids are aliphatic or aromatic acids of the aforementioned type. Particular preference is given to adipic acid, isophthalic acid and optionally trimellitic acid, very particular preference to adipic acid.

Examples of hydroxycarboxylic acids that may be used as reaction participants in the preparation of a polyester polyol having terminal hydroxyl groups include hydroxycaproic acid, hydroxybutyric acid, hydroxydecanoic acid, hydroxystearic acid and the like. Suitable lactones are caprolactone, butyrolactone and homologues. Preference is given to caprolactone.

In component f), it is also possible to use polycarbonates having hydroxyl groups, preferably polycarbonatediols, having number-average molecular weights Mₙ of 400 to 8000 g/mol, preferably of 600 to 3000 g/mol. These are obtainable by reaction of carbonic acid derivatives, such as diphenyl carbonate, dimethyl carbonate or phosgene, with polyols, preferably diols.

Examples of such diols are ethylene glycol, propane-1,2- and 1,3-diol, butane-1,3- and 1,4-diol, hexane-1,6-diol, octane-1,8-diol, neopentyl glycol, 1,4-bishydroxymethyl-cyclohexane, 2-methylpropane-1,3-diol, 2,2,4-trimethylpentane-1,3-diol, dipropylene glycol, polypropylene glycols, dibutylene glycol, polybutylene glycols, bisphenol A, and lactone-modified diols of the aforementioned type. The polycarbonates having hydroxyl groups preferably have a linear structure.

In a preferred embodiment of the invention, the polyurethane urea used in accordance with the invention is formed from
a) at least one of an aliphatic, an araliphatic and a cycloaliphatic diisocyanate having at least one isocyanate group bonded to a secondary or tertiary carbon atom,
b) at least one polyether polyol having a number-average molecular weight Mₙ of ≧500 and ≦2500 g/mol and a hydroxyl functionality of ≧1.9 and ≦3,
c) at least one amino-functional compound having at least two (isocyanate-reactive) amino groups and selected from ethylenediamine, IPDA and/or H12-MDA,
d) optionally, at least one alcohol having at least two hydroxyl groups and a molar mass of ≧60 and ≦399 g/mol,
e) optionally, at least one compound having a group reactive toward isocyanate groups and
f) optionally, ≦20% by weight, based on the total mass of the polyurethane urea, of at least one different polyol than b) having a number-average molecular weight Mₙ of ≧500 and ≦6000 g/mol and a hydroxyl functionality of ≧1.5 and ≦4.

Further preferably, the polyurethane urea, in this aforementioned embodiment, is formed from ≧5% and ≦60% by weight of component a), ≧30% and ≦90% by weight of component b), ≧2% and ≦25% by weight of component c), ≧0% and ≦10% by weight of component d), ≧0% and ≦10% by weight of component e) and ≧0% and ≦20% by weight of component f), based in each case on the total mass of the polyurethane urea, where components a) to f) add up to 100% by weight.

Especially preferably, the polyurethane urea, in this aforementioned embodiment, is formed from ≧10% and ≦40% by weight of component a), ≧55% and ≦85% by weight of component b), ≧5% and ≦20% by weight of component c), ≧0% and ≦3% by weight of component d), ≧0% and ≦3% by weight of component e) and ≧0% and ≦1% by weight of component f), based in each case on the total mass of the polyurethane urea, where components a) to f) add up to 100% by weight.

In a particularly preferred embodiment of the invention, the polyurethane urea used in accordance with the invention is formed from
a) at least one isocyanate selected from IPDI and H12-MDI,
b) at least one polyether polyol having a number average molecular weight Mₙ≧500 and ≦2500 and a hydroxyl functionality of ≧1.9 and ≦3, selected from polypropylene glycols and/or poly(tetramethylene glycol) polyether polyols,
c) at least one amino-functional compound selected from IPDA and H12-MDA,
d) optionally at least one alcohol having at least two hydroxyl groups and a molar mass of ≧60 and ≦399 g/mol,
e) optionally at least one compound having a group reactive toward isocyanate groups and
f) optionally ≦20% by weight, based on the total mass of the polyurethane urea, of at least one different polyol than b) having a number-average molecular weight Mₙ of ≧500 and ≦6000 g/mol and a hydroxyl functionality of ≧1.5 and ≦4.

Further preferably, the polyurethane urea, in this aforementioned embodiment, is formed from ≧5% and ≦60% by weight of component a), ≧30% and ≦90% by weight of component b), ≧2% and ≦25% by weight of component c), ≧0% and ≦10% by weight of component d), ≧0% and ≦10% by weight of component e) and ≧0% and ≦20% by weight of component f), based in each case on the total mass of the polyurethane urea, where components a) to f) add up to 100% by weight.

Especially preferably, the polyurethane urea, in this aforementioned embodiment, is formed from ≧10% and ≦40% by weight of component a), ≧55% and ≦85% by weight of component b), ≧5% and ≦20% by weight of component c), ≧0% and ≦3% by weight of component d), ≧0% and ≦3% by weight of component e) and ≧0% and ≦1% by weight of component f), based in each case on the total mass of the polyurethane urea, where components a) to f) add up to 100% by weight.

Preferably, the polyurethane urea is formed from components a) to c) and optionally d) to f), more preferably from components a) to c).

Advantageously, the polyurethane urea has a number-average molecular weight Mₙ≧2000 and ≦50 000 g/mol, particularly advantageously ≧3000 and ≦20 000 g/mol.

The polyurethane urea is preferably prepared by reacting components a) and b) and optionally d) and f) in a first step to give an NCO-terminated prepolymer, which is then reacted in a subsequent step with component c) and optionally components d) and e).

For the preparation of the polyurethane ureas, preferably, components a) and b) and optionally d) and f) for preparation of an NCO-terminated prepolymer are initially charged in full or in part, optionally diluted with a solvent inert toward isocyanate groups, and heated up to temperatures in the range from 50 to 120° C. The isocyanate addition reaction can be accelerated using the catalysts known in polyurethane chemistry. A preferred variant, however, works without the addition of urethanization catalysts.

Subsequently, any constituents of a) and b) and optionally d) and f) which have not yet been added at the start of the reaction are metered in.

In the preparation of the NCO-terminated prepolymers from components a) and b) and optionally d) and f), the molar ratio of isocyanate groups to isocyanate reactive groups is generally ≧1.05 and ≦3.5, preferably ≧1.1 and ≦3.0, more preferably ≧1.1 and ≦2.5.

Isocyanate-reactive groups are understood to mean all groups reactive toward isocyanate groups, for example primary and secondary amino groups, hydroxyl groups or thiol groups.

The conversion of components a) and b) and optionally d) and f) to the prepolymer is effected in part or in full, but preferably in full. In this way, polyurethane prepolymers containing free isocyanate groups are obtained in substance or in solution. Preferably, the NCO-terminated prepolymer is prepared from components a) and b).

Thereafter, preferably, in a further process step, if this has been done only partly, if at all, the prepolymer obtained is dissolved with the aid of one or more organic solvents. The solvent used is preferably likewise a solvent or solvent mixture, where the solvent consists of one or more monohydroxy-functional alcohols or a solvent mixture consisting of organic solvents containing ≧50% by weight, based on the total mass of the solvent mixture, of at least one monohydroxy-functional alcohol is used. In respect of the solvent and solvent mixture, the preferred embodiments below relating to the solvent or solvent mixture in which the polyurethane urea is dissolved are likewise applicable. The solvent or solvent mixture may also be different than the solvent or solvent mixture in which the polyurethane urea as end product is dissolved at a later stage. The solvent or solvent mixture is preferably identical to the solvent or solvent mixture in which the polyurethane urea as end product is dissolved at a later stage.

Preferably, the solvent used in the preparation consists of one or more monohydroxy-functionalized alcohols.

The ratio of solvent to prepolymer is preferably ≧1:10 and ≦5:1, more preferably ≧1:2 and ≦2:1, parts by weight.

Prior to the dissolution, the prepolymer is cooled down to temperatures of −20 to 60° C., preferably 0 to 50° C. and more preferably 15 to 40° C.

In a further step that optionally follows the dissolution of the NCO-terminated prepolymer, the NCO-terminated prepolymer obtained in the first step is then preferably reacted fully or partly with component c) and optionally components d) and e). This reaction is generally referred to as chain extension, or in the case of component e) as chain termination.

Preference is given here to initially charging the NCO-terminated prepolymer, and metering in components c) and optionally d) and e). Preference is given to firstly partly reacting the NCO groups of the prepolymer with components c) and optionally d), followed by chain termination by reaction of the remaining NCO groups with component e). Components c) and optionally e) may also be added stepwise in two or more steps, especially in two steps.

Component c) and optionally d) and e) are preferably used dissolved in one or more organic solvents. The solvent used is preferably likewise a solvent or solvent mixture, where the solvent consists of one or more monohydroxy-functional alcohols or a solvent mixture consisting of organic solvents containing ≧50% by weight, based on the total mass of the solvent mixture, of at least one monohydroxy-functional alcohol is used. In respect of the solvent and solvent mixture, the preferred embodiments below relating to the solvent or solvent mixture in which the polyurethane urea is dissolved are likewise applicable.

The solvent or solvent mixture may also be different than the solvent or solvent mixture in which the polyurethane urea as end product is dissolved at a later stage. The solvent or solvent mixture is preferably identical to the solvent or solvent mixture in which the polyurethane urea as end product is dissolved at a later stage.

Preferably, the solvent used in the preparation for component c) consists of one or more monohydroxy-functionalized alcohols.

When solvents are used as diluents, the diluent content in the components c) used in the chain extension, and optionally d) and e), is preferably 1% to 95% by weight, preferably 3% to 50% by weight, based on the total weight of component c) and optionally d) and e) including diluents.

Components c) and optionally d) and e) are preferably added at temperatures of −20 to 60° C., preferably 0 to 50° C. and more preferably of 15 to 40° C.

The degree of chain extension, i.e. the molar ratio of NCO-reactive groups of the components c) used for chain extension and chain termination, and optionally d) and e), to free NCO groups of the prepolymer, is generally ≧50 and ≦120%, more preferably ≧60 and ≦100% and most preferably ≧70 and ≦95%.

Preferably, the molar ratio of isocyanate-reactive groups of component c) to the free NCO groups of the prepolymer is ≧50% and ≦120%, more preferably ≧60% and ≦100% and most preferably ≧70% and ≦95%.

In a preferred embodiment of the invention, the free NCO groups of the prepolymer are only partly reacted with component c), the molar ratio of isocyanate-reactive groups of component c) to the free NCO groups of the prepolymer preferably being ≧60% and ≦95% and the remaining free NCO groups being depleted by reaction with the hydroxyl groups of the solvent, so as to form an NCO-free polyurethane urea.

After the preparation, the polyurethane urea, if solvents or solvent mixtures of the invention have already been used in the preparation process, can still be diluted and dissolved with a solvent or solvent mixture, in which case the solvent consists of one or more monohydroxy-functional alcohols or a solvent mixture consisting of organic solvents containing ≧50% by weight, based on the total mass of the solvent mixture, of at least one monohydroxy-functional alcohol is used.

If no solvents or solvent mixtures have been used during the reaction, after the polyurethane urea has been prepared, it is used in a solvent or solvent mixture, in which case the solvent consists of one or more monohydroxy-functional alcohols or a solvent mixture consisting of organic solvents and containing ≧50% by weight, based on the total mass of the solvent mixture, of at least one monohydroxy-functional alcohol is used.

The dissolution of the polyurethane urea can be effected by standard techniques for shearing, for example by stirring with standard stirrers as specified in DIN 28131.

The polyurethane urea is preferably dissolved without the additional addition of external emulsifiers. The polyurethane urea solutions used in accordance with the invention preferably do not comprise any external emulsifiers.

Suitable solvents or constituents of the solvent mixture are in principle all monohydroxy-functional aliphatic alcohols having one to six carbon atoms, for example methanol, ethanol, n-propanol, isopropanol, n-butanol, sec-butanol and/or butylglycol. More preferably, the monohydroxy-functional alcohol is ethanol. If a solvent mixture is used, as well as the monohydroxy-functional alcohols, it is also possible to use ≦50% by weight, based on the total mass of the solvent mixture, of a further organic solvent. Suitable solvents here are, for example, esters, for example ethyl acetate, butyl acetate, methoxypropyl acetate or butyrolactone, ketones, for example acetone or methyl ethyl ketone, ethers, for example tetrahydrofuran or tert-butyl methyl ether, aromatic solvents, for example xylene or solvent naphtha. In the case of use of ethanol, typical denaturing agents may be present as additives in the customary added amounts.

Preferably, the proportion of the further organic solvents is ≦30% by weight, more preferably ≦5% by weight and most preferably ≦2% by weight. In a most preferred embodiment, no further organic solvents are present aside from monohydroxy-functional aliphatic alcohols.

Unsuitable further solvents are physiologically incompatible solvents, for example dimethylformamide, N-methylpyrrolidone or toluene, as often used as co-solvents for polyurethanes or polyurethane ureas, these should preferably not be present in cosmetic compositions.

The further solvents are not water. The polyurethane urea solution obtained by dissolving the polyurethane urea in the solvents or solvent mixtures used in accordance with the invention is preferably anhydrous, excluding the proportions of water present as a result of the preparation in the organic solvents used.

The water content of the polyurethane urea solution is ≦10% by weight, preferably ≦4.5% by weight and most preferably ≦1% by weight, based on the total mass of the polyurethane urea solution.

The proportion of the polyurethane urea (as active substance) in the polyurethane urea solution used in accordance with the invention (also referred to as solids content) is preferably ≧10% and ≦80% by weight, more preferably ≧15% and ≦60% by weight and most preferably ≧20% and ≦50% by weight.

The invention further provides the sunscreen composition of the invention for protection of the skin and hair from adverse effects of solar radiation.

The sunscreen compositions of the invention are preferably in the form of gels, oils, sprays or aerosols, which are preferably transparent. “Transparent” in the context of the present invention means that the turbidity values of the composition are ≦100 NTU (Nephelometric Turbidity Unit), preferably ≦50 NTU, more preferably ≦10 NTU and most preferably ≦5 NTU. Turbidity values are determined by a scattered light measurement at a 90° angle (nephelometry) at a measurement radiation wavelength of 860 nm in accordance with DIN EN ISO 7027, conducted at 23° C. with a model 2100AN laboratory turbidimeter from HACH LANGE GmbH, Berlin, Germany.

The sunscreen compositions can also be foamed with a propellant gas.

The proportion of the polyurethane urea solution used in the sunscreen composition is preferably ≧0.5% and ≦80% by weight, more preferably ≧1% and ≦60% by weight and most preferably ≧2% and ≦40% by weight, based on the total mass of the sunscreen composition.

The solids content of the polyurethane urea solution is preferably chosen such that the cosmetic compositions contain preferably ≧0.1% and ≦30% by weight, more preferably ≧0.5% and ≦20% by weight and most preferably ≧1% and ≦10% by weight of the polyurethane urea as active substance, based on the total mass of the sunscreen composition.

Active substance is understood to mean the polyurethane urea without solvent or solvent mixture.

The sunscreen compositions of the invention preferably have a viscosity of ≧2 and ≦20 000 mPas. Compositions in the form of gels or lotions more preferably have a viscosity of ≧1000 and ≦20 000 mPas and most preferably of ≧2000 and ≦10 000 mPas. Sprayable compositions such as sun sprays more preferably have a viscosity of ≧2 and ≦2000 mPas and most preferably of ≧5 and ≦500 mPas.

The viscosities reported are determined by means of rotary viscometry to DIN 53019 at 23° C. with a rotary viscometer from Anton Paar Germany GmbH, Ostfildern, DE, at a shear rate of 10 s⁻¹.

The sunscreen compositions of the invention may be present in different consistency: in semisolid form, especially as gels, or in mobile form, especially as compositions, aerosols or oils.

Preferably, the sunscreen compositions are those that are predominantly alcohol-based, i.e. contain ≧10% and ≦90% by weight, based on the total mass of the cosmetic composition, preferably ≧15% and ≦70% by weight and more preferably ≧20% and ≦60% by weight of aliphatic alcohols having 1 to 6 carbon atoms, preferably 1 to 4 carbon atoms. The alcohols are preferably selected from ethanol and isopropanol; polyol and derivatives thereof, such as propylene glycol, dipropylene glycol, butylene 1,3-glycol, polypropylene glycol, glycol ethers such as alkyl (C1-4) ethers of mono-, di- or tripropylene glycol or mono-, di- or triethylene glycol, or mixtures thereof. More preferably, the alcohols contain ethanol or consist thereof; most preferably, the alcohol used is ethanol.

More preferably, the sunscreen compositions are alcoholic solutions.

The sunscreen compositions preferably contain a water content of ≧0% and ≦30% by weight, more preferably ≧0% and ≦20% by weight, even more preferably of ≧0% and ≦5% by weight and further preferably of ≧0% and ≦2% by weight. Especially preferably, the sunscreen compositions are anhydrous, and thus contain no more water than what is unavoidably introduced into the formulation via the raw materials as a result of production.

The water used in the composition according to the invention may be a blossom water, pure demineralized water, mineral water, thermal water and/or seawater.

The sunscreen composition of the invention preferably contains a total of ≧1% and ≦40% by weight, more preferably ≧5% and ≦35% by weight and most preferably ≧10% and ≦30% by weight of sunscreen filter substances, based on the total weight of the sunscreen composition. The stated amount is the sum total of all the sunscreen filter substances present in the sunscreen composition of the invention. Sunscreen filter substances can also be referred to as sunscreen filters or sunscreen-imparting substances.

The sunscreen filter is especially UV filters which filter light in the UV wavelength region, especially of less than 400 nm. The terms “sunscreen filter substances” and “UV filter substances” are used as equivalent terms in the context of this application.

Typically, the UV wavelength range is subdivided as follows:

| UV light | Wavelength range in nm | |
|---|---|---|
| Near UV | 400-200 | nm |
| UV-A | 380-315 | nm |
| UV-B | 315-280 | nm |
| UV-C | 280-100 | nm |
| Far UV, | 200-10 | nm |
| vacuum radiation | | |
| Extreme UV | 31-1 | nm |

The sunscreen filters (or UV filters) may be selected from the organic filters, the physical filters and/or mixtures thereof.

The sunscreen composition of the invention may especially comprise UV-A filters, UV-B filters, broadband filters and/or physical filters as sunscreen filter substances. The sunscreen composition of the invention preferably contains mixtures of at least two of these aforementioned types of sunscreen filter substances. The sunscreen composition of the invention may also contain two or more sunscreen filter substances that can be assigned to one of these two types of sunscreen filter substances, i.e., for example, two or more UV-A filters and/or two or more UV-B filters. Any desired combinations are possible.

The UV filters used may be oil-soluble or water-soluble. The list of UV filters mentioned which follows is of course nonlimiting.

Examples of UV-B filters include:
(1) salicylic acid derivatives, particularly homomenthyl salicylate, octyl salicylate and 4-isopropylbenzyl salicylate;
(2) cinnamic acid derivatives, especially 2-ethylhexyl p-methoxycinnamate, available from Givaudan under the Parsol MCX name, and isopentyl 4-methoxycinnamate;
(3) liquid β,β′-diphenylacrylate derivatives, especially 2-ethylhexyl α,β′-diphenylacrylate or octocrylene, available from BASF under the UVINUL N539 name;
(4) p-aminobenzoic acid derivatives, especially 2-ethylhexyl 4-(dimethylamino)benzoate, amyl 4-(dimethylamino)benzoate;
(5) 3-benzylidenecamphor derivatives, especially 3-(4-methylbenzyl-idene)camphor, commercially available from Merck under the EUSOLEX 6300 name, 3-benzylidenecamphor, benzylidenecamphorsulfonic acid and polyacrylamidomethylbenzylidenecamphor;
(6) 2-phenylbenzimidazole-5-sulfonic acid, available under the EUSOLEX 232 name from Merck; or 2-phenylbenzimidazole-5-sulfonic acid that has been neutralized by organic amines, as described in DE 20 2010 006 005 U1;
(7) 1,3,5-triazine derivatives, especially: -2,4,6-tris[p-(2′-ethylhexyl-1′-oxycarbonyl)anilino]-1,3,5-triazine, supplied by BASF under the UVINUL T150 name, and -dioctylbutamidotriazone, supplied by Sigma 3V under the UVASORB HEB name;
(8) esters of benzalmalonic acid, especially di(2-ethylhexyl) 4-methoxybenzalmalonate and 3-(4-(2,2-bisethoxycarbonyl-vinyl)phenoxy)propenyl)methoxysiloxane/dimethylsiloxane copolymer, available from Roche Vitamins under the PARSOL SLX name; and
(9) the mixtures of these filters.

More preferably, the sunscreen compositions exhibit an SPF-boosting effect, meaning that the sunscreen composition of the invention comprising the polyurethane solution of the invention and sunscreen filter substances has a distinctly higher SPF than the mixture of the sunscreen filter substances alone.

The sunscreen composition of the invention preferably contains at least one UV-B filter, more preferably octocrylene, in an amount of ≧4% and ≦12% by weight, preferably

≧5% and ≦12% by weight, more preferably ≧6% and ≦12% by weight, most preferably ≧7% and ≦11% by weight, based on the total weight of the sunscreen composition.

Examples of UV-A filters include:
(1) dibenzoylmethane derivatives, particularly 4-(t-butyl)-4′-methoxydibenzoylmethane, which is supplied by Givaudan under the PARSOL 1789 name, and 1-phenyl-3-(4′-isopropylphenyl)propane-1,3-dione;
(2) benzene-1,4-[di(3-methylidenecamphor-10-sulfonic acid)], optionally fully or partly neutralized, commercially available under the MEXORYL SX name from Chimex.
(3) hexyl 2-(4′-diethylamino-2′-hydroxybenzoyl)benzoate (also aminobenzophenone);
(4) silane derivatives or polyorganosiloxanes having benzophenone groups;
(5) anthranilates, particularly menthyl anthranilate, which is supplied by Symrise under the NEO HELIOPAN MA name;
(6) compounds containing at least two benzazolyl groups or at least one benzodiazolyl group per molecule, especially 1,4-bis(benzimidazolyl)-phenylene-3,3′,5,5′-tetrasulfonic acid and salts thereof, available from Symrise;
(7) silicon derivatives of benzimidazolylbenzazoles that are N-substituted, or of benzofuranylbenzazoles, especially:
   2-[1-[3-[1,3,3,3-tetramethyl-1-[(trimethylsilyl)oxy]disiloxanyl]propyl]-1H-benzimidazol-2-yl]benzoxazole;
   2-[1-[3-[1,3,3,3-tetramethyl-1-[(trimethylsilyl)oxy]disiloxanyl]propyl]-1H-benzimidazol-2-yl]-benzothiazole;
   2-[1-(3-trimethylsilanylpropyl)-1H-benzimidazol-2-yl]benzoxazole;
   6-methoxy-1,1′-bis(3-trimethylsilanylpropyl)1H,1′H-[2,2′]dibenzimidazolylbenzoxazole;
   2-[1-(3-trimethylsilanylpropyl)-1H-benzimidazol-2-yl]benzothiazole;
which are described in patent application EP-A-1 028 120;
(8) triazine derivatives, especially 2,4-bis[5-1(dimethylpropyl)benzoxazol-2-yl-(4-phenyl)-imino]-6-(2-ethylhexyl)-imino-1,3,5-triazine, supplied by 3V under the UVASORBK2A name [CAS Nr. 288254-16-0]; and
(9) mixtures thereof.

In a preferred embodiment, the sunscreen composition of the invention comprises, as sunscreen filter substances, preferably at least one UV-a filter which is preferably a dibenzoylmethane derivative, more preferably 4-(t-butyl)-4′-methoxydibenzoylmethane. This dibenzoylmethane derivative, preferably 4-(t-butyl)-4′-methoxydibenzoylmethane, is preferably present in the sunscreen composition of the invention in an amount of ≧1% and ≦5% by weight, based on the total weight of the sunscreen composition.

Examples of suitable broadband filters include:
(1) benzophenone derivatives, for example
   2,4-dihydroxybenzophenone (benzophenone-1);
   2,2′,4,4′-tetrahydroxybenzophenone (benzophenone-2);
   2-hydroxy-4-methoxybenzophenone (benzophenone-3), available from BASF under the UNIVNUL M40 name;
   2-hydroxy-4-methoxybenzophenone-5-sulfonic acid (benzophenone-4), and the sulfonate form thereof (benzophenone-5), available from BASF under the UVINUL MS40 name;
   2,2′-dihydroxy-4,4′-dimethoxybenzophenone (benzophenone-6);
   5-chloro-2-hydroxybenzophenone (benzophenone-7);
   2,2′-dihydroxy-4-methoxybenzophenone (benzophenone-8);
   the disodium salt of 2,2′-dihydroxy-4,4′-dimethoxybenzophenone-5,5′-disulfonic acid (benzophenone-9);
   2-hydroxy-4-methoxy-4′-methylbenzophenone (benzophenone-10);
   benzophenone-11;
   2-hydroxy-4-(octyloxy)-benzophenone (benzophenone-12).
(2) triazine derivatives, especially 2,4-bis{[4-2-ethylhexyloxy)-2-hydroxy]phenyl}-6-(4-methoxyphenyl)-1,3,5-triazine, which is supplied by Ciba Geigy under the TINOSORB S name, and 2,2′-methylenebis[6-(2H-benzotriazol-2-yl)-4-(1,1,3,3-tetramethylbutyl)phenol], available from Ciba Geigy under the TINOSORB M name; and
(3) 2-(1H-benzotriazol-2-yl)-4-methyl-6-[2-methyl-3-[1,3,3,3-tetramethyl-1-[(trimethylsilyl)oxy]disiloxanyl]propyl]phenol with the INCI name drometrizole trisiloxane.

It is also possible to use a mixture of two or more filters and a mixture of UV-B filters, UV-A filters and broadband filters, and also mixtures with physical filters.

The physical filters may include, for example, the sulfates of barium, and oxides of titanium (titanium dioxide, amorphous or crystalline in the form of rutile and/or anatase), of zinc, of iron, of zirconium, of cerium, of silicon, of manganese or mixtures thereof. The metal oxides may be in particle form with a size in the micrometer range or nanometer range (nanopigments). The mean particle sizes for the nanopigments are, for example, 5 to 100 nm. In transparent compositions, preference is given to using physical filters having particle sizes in the nanometer range or no physical filters.

Oil-soluble UV filters may be those that are liquid, especially oil-like, and may themselves also serve as solvents for other oil-soluble UV filters or those that are solid and are used dissolved in oils.

Liquid oil-soluble UV filters used with preference are octocrylene, ethylhexyl methoxycinnamate, ethylhexyl salicylate and homosalate.

Solid oil-soluble UV filters used with preference are butylmethoxydibenzoylmethane (Avobenzone), dioctylbutylamidotriazone (INCI: diethylhexyl butamidotriazone), 2,4-bis{[4-(2-ethylhexyloxy)-2-hydroxy]phenyl}-6-(4-methoxyphenyl)-1,3,5-triazine (INCI: bis-ethylhexyloxyphenol methoxyphenyl triazine), ethylhexyltriazone, diethylamino hydroxybenzoyl hexyl benzoate. In addition, it is also possible to use all other oil-soluble filters listed in Annex VI of the EU Cosmetics Directive (1223/2009).

In a preferred embodiment of the invention, the cosmetic compositions comprise at least one liquid oil-soluble UV filter.

In a preferred embodiment of the sunscreen composition of the invention, it contains ≧5% and ≦35% by weight of sunscreen filter substances, based on the total weight of the sunscreen composition, of which at least one of the sunscreen filter substances is benzophenone, a benzophenone derivative or a triazine-derived derivative or octocrylene and the sunscreen filter substances make up preferably ≧4% and ≦12% by weight of the total weight of the sunscreen composition. More preferably, at least one of the sunscreen filter substances is octocrylene.

The sunscreen compositions of the invention, in preferred embodiments, have a sun protection factor (SPF) of more than 15, preferably of more than 20, measured by the International Sun Protection Factor (SPF) test method of COLIPA. This test method is known to those skilled in the art.

The sunscreen compositions of the invention may further comprise oils and/or waxes, and the oils may be non-volatile oils and/or volatile oils.

The sunscreen composition of the invention advantageously contains ≧0% and ≦45% by weight of oils, based on the total weight of the composition, and particularly advantageously ≧0.01% and ≦45% by weight of oils and very particularly advantageously ≧0.1% and ≦20% by weight of oils.

The non-volatile oils are advantageously chosen from the group of mineral, animal, plant or synthetic origin, polar and/or nonpolar oils or mixtures thereof.

The polar oils are advantageously chosen from the group of:

a) esters of saturated and unsaturated, branched and/or unbranched alkylcarboxylic acids of chain length from 3 to 30 carbon atoms and saturated and unsaturated, branched and/or unbranched alcohols of chain length from 3 to 30 carbon atoms,
b) esters of aromatic carboxylic acids and saturated and unsaturated, branched and/or branched alcohols of chain length from 3 to 30 carbon atoms.

Such ester oils can then advantageously be chosen from the group of:

isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, isotridecyl isononanoate, 2-ethylhexyl palmitate, 2-ethylhexyl laurate, 2-ethylhexyl isostearate, 2-hexyldecyl stearate, 2-octyldodecyl palmitate, 2-ethylhexyl cocoate, oleyl oleate, oleyl erucate, erucyl oleate, erucyl erucate, dicaprylyl carbonate (CETIOL CC) and cocoglycerides (MYRITOL 331), and also synthetic, semisynthetic and natural mixtures of such esters, for example jojoba oil.
c) alkyl benzoates C₁₂₋₁₅-alkyl benzoate (FINSOLV TN from Finetex) or 2-phenylethyl benzoate (X-TEND 226 from ISP)
d) lecithins and the fatty acid triglycerides, namely the triglyceryl esters of saturated and unsaturated, branched and/or unbranched alkanecarboxylic acids of chain length 8 to 24, especially 12 to 18, carbon atoms. For example, the fatty acid triglycerides may be chosen from the group of cocoglyceride, olive oil, sunflower oil, soybean oil, groundnut oil, rapeseed oil, almond oil, palm oil, coconut oil, castor oil, wheatgerm oil, grapeseed oil, safflower oil, evening primrose oil, macadamia nut oil, apricot kernel oil, avocado oil and the like.
e) the dialkyl ethers and dialkyl carbonates, advantageous examples being dicaprylyl ether (CETIOL OE from Cognis) and/or dicaprylyl carbonate (for example CETIOL CC from Cognis).
f) saturated or unsaturated, branched or unbranched alcohols, for example octyldodecanol.

The non-volatile oil may likewise advantageously also be a nonpolar oil which is chosen from the group of a branched and unbranched hydrocarbons, especially mineral oil, vaseline oil, paraffin oil, squalane and squalene, polyolefins, for example polydecenes, hydrogenated polyisobutenes, C₁₃₋₁₆ isoparaffin and isohexadecane.

The nonpolar non-volatile oil may also be selected from the non-volatile silicone oils.

The non-volatile silicone oils may include the polydimethylsiloxanes (PDMS) that are optionally phenylated, such as phenyltrimethicone, or are optionally substituted by aliphatic and/or aromatic groups or by functional groups, for example hydroxyl groups, thiol groups and or amino groups; polysiloxanes modified with fatty acids, fatty alcohols or polyoxyalkylenes, and mixtures thereof.

Particularly advantageous oils are 2-ethylhexyl isostearate, octyldodecanol, isotridecyl isononanoate, isoeicosane, 2-ethylhexyl cocoate, C12-15 alkyl benzoate, caprylic/capric triglyceride, dicaprylyl ether, mineral oil, dicaprylyl carbonate, cocoglyceride, butylene glycol dicaprylate/dicaprate, hydrogenated polyisobutene, cetaryl isononanoate, isodecyl neopentanoate, squalane, C₁₃₋₁₆ isoparaffin.

The sunscreen composition of the invention may further comprise a wax.

In the context of the present document, a wax is defined as a lipophilic fatty substance which is solid at room temperature (25° C.) and shows a reversible solid/liquid change of state at a melting temperature of 30° C. to 200° C. The wax is advantageously chosen from the group of natural waxes, for example cotton wax, carnauba wax, candelilla wax, esparto wax, japan wax, montan wax, sugarcane wax, beeswax, wool wax, shellac, microwaxes, ceresin, ozokerite, ouricury wax, cork fiber wax, lignite waxes, berry wax, shea butter, or synthetic waxes such as paraffin waxes, polyethylene waxes, waxes produced by Fischer-Tropsch synthesis, hydrogenated oils, fatty acid esters and glycerides that are solid at 25° C., silicone waxes and derivatives (alkyl derivatives, alkoxy derivatives and/or esters of polymethylsiloxane) and mixtures thereof. The waxes may take the form of stable dispersions of colloidal wax particles which can be produced by known methods, for example according to “Microemulsions Theory and Practice”, L. M. Prince Ed., Academic Press (1977), pages 21-32.

The waxes may be present in amounts of ≧0% and ≦10% by weight, based on the total weight of the composition, and preferably ≧0.01% and ≦10% by weight and most preferably ≧0.1% and ≦5% by weight.

The sunscreen composition of the invention may further comprise a volatile oil which is selected from the group of volatile hydrocarbons, siliconized oils and fluorinated oils.

The volatile oils may be present in amounts of ≧0% and ≦25% by weight, based on the total weight of the composition, preferably ≧0% and ≦20% by weight and more preferably ≧0.1% and ≦15% by weight.

In the context of the present document, a volatile oil is an oil which evaporates within less than one hour on contact with the skin at room temperature and atmospheric pressure. The volatile oil is liquid at room temperature and, at room temperature and atmospheric pressure, has a vapor pressure of 0.13 to 40 000 Pa (10⁻³ to 300 mg Hg), preferably 1.3 to 13 000 Pa (0.01 to 100 mm Hg) and more preferably 1.3 to 1300 Pa (0.01 to 10 mm Hg), and a boiling point of 150 to 260° C. and preferably 170 to 250° C.

A hydrocarbon oil is understood to mean an oil which is formed essentially from carbon atoms and hydrogen atoms, with or without oxygen atoms or nitrogen atoms, and does not contain any silicon atoms or fluorine atoms, and it may also consist of carbon atoms and hydrogen atoms; it may contain ester groups, ether groups, amino groups or amide groups.

A siliconized oil is understood to mean an oil containing at least one silicon atom and especially Si—O groups.

A fluorinated oil is understood to mean an oil containing at least one fluorine atom.

The hydrocarbon oil which is volatile in accordance with the invention may be selected from the hydrocarbons having a flashpoint of 40 to 102° C., preferably 40 to 55° C. and even more preferably 40 to 50° C.

Preferably, the volatile hydrocarbon oils are those having 8 to 16 carbon atoms and mixtures thereof, especially branched C₈₋₁₆-alkanes such as the isoalkanes (which are also referred to as isoparaffins) having 8 to 16 carbon atoms, isododecane, isodecane, isohexadecane and, for example, the oils that are supplied under the ISOPARS or PERMETYLS trade names; and the branched C₈₋₁₆ esters, such as isohexyl neopentanoate and mixtures thereof.

Particularly advantageous are the volatile hydrocarbon oils such as isododecane, isodecane and isohexadecane.

The siliconized oil which is volatile in accordance with the invention may be selected from the siliconized oils having a flashpoint of 40 to 102° C., preferably a flashpoint exceeding 55° C. and not more than 95° C. and more preferably in the range from 65 to 95° C.

For example, the volatile siliconized oils are straight-chain or cyclic silicone oils having 2 to 7 silicon atoms, wherein the silicones optionally containing alkyl or alkoxy groups having 1 to 10 carbon atoms.

Particularly advantageous are the volatile siliconized oils such as octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane and mixtures thereof.

The volatile fluorinated oil generally does not have a flashpoint.

For example, the volatile fluorinated oils are nonafluoroethoxybutane, nonafluoromethoxybutane, decafluoropentane, tetradecafluorohexane, dodecafluoropentane and mixtures thereof.

The sunscreen compositions of the invention may additionally comprise one or more further additives that are customary in cosmetics, such as auxiliary and additions, for example emulsifiers, interface-active substances, defoamers, thickeners, surfactants, humectants, filler, film former, solvent, coalescent, gel former and/or other polymer dispersions, for example dispersions based on polyacrylates, fillers, plasticizers, pigments, dyes, leveling agents, thixotropic agents, sleekness agents, preservatives, sensory additive, propellant gas, for example propane/butane or dimethyl ether, etc. The amounts of the various additions are known to the person skilled in the art for the range to be used and are preferably in the range of ≧0% and ≦40% by weight, preferably ≧0.01% and ≦40% by weight, based on the total weight of the sunscreen composition.

The sunscreen composition of the invention may comprise one or more humectants (moisturizers). Particularly advantageous humectants in the context of the present invention are, for example, glycerol, polyglycerol, sorbitol, dimethyl isosorbide, lactic acid and/or lactates, especially sodium lactate, butylene glycol, propylene glycol, biosaccharide gum-1, glycine soya, hydroxyethylurea, ethylhexyloxyglycerol, pyrrolidonecarboxylic acid and urea. In addition, it is especially advantageous to use polymeric moisturizers from the group of the water-soluble and/or water-swellable polysaccharides and/or those that can be gelated with the aid of water. Especially advantageous are, for example, hyaluronic acid, chitosan and/or a fucose-rich polysaccharide available under the FUCOGEL 1000 name from SOLABIA S.A.

In a preferred embodiment of the invention, the sunscreen composition comprises
I) at least one polyurethane urea dissolved in a solvent or solvent mixture, where the solvent consists of one or more monohydroxy-functional alcohols or a solvent mixture consisting of organic solvents containing ≧50% by weight, based on the total mass of the solvent mixture, of at least one monohydroxy-functional alcohol is used,
II) at least one oil-soluble sunscreen filter substance,
III) at least one aliphatic alcohol having 1 to 6 carbon atoms.

In a particularly preferred embodiment of the invention, the sunscreen composition comprises
I) ≧0.5% and ≦80% by weight of at least one polyurethane urea dissolved in a solvent or solvent mixture, where the solvent consists of one or more monohydroxy-functional alcohols or a solvent mixture consisting of organic solvents containing ≧50% by weight, based on the total mass of the solvent mixture, of at least one monohydroxy-functional alcohol is used,
II) ≧1% and ≦40% by weight of sunscreen filter substances, comprising at least one oil-soluble sunscreen filter substance,
III) ≧10% and ≦90% by weight of at least one aliphatic alcohol having 1 to 6 carbon atoms,
IV) ≧0% and ≦40% by weight of at least one oil and/or ≧0% and ≦10% by weight of at least one wax, and
V) ≧0% and ≦40% by weight of further additives customary for sunscreen compositions,
based in each case on the total mass of the sunscreen composition, where components I) to V) add up to 100% by weight.

Component IV) is preferably equal to zero only when component B) already contains at least one liquid oil-soluble sunscreen filter substance. The latter can then be regarded as an oil which is also able to dissolve further solid oil-soluble sunscreen filter substances.

The solids content of the polyurethane urea solution is preferably chosen such that the sunscreen composition contains preferably ≧0.5% and ≦20% by weight of the polyurethane urea as active substance, based on the total mass of the cosmetic formulation.

In a very particularly preferred embodiment of the invention, the sunscreen composition comprises
I) ≧2% and ≦40% by weight of at least one polyurethane urea dissolved in a solvent or solvent mixture, where the solvent consists of one or more monohydroxy-functional alcohols or a solvent mixture consisting of organic solvents containing ≧50% by weight, based on the total mass of the solvent mixture, of at least one monohydroxy-functional alcohol is used,
II) ≧10% and ≦30% by weight of sunscreen filter substances, comprising at least one oil-soluble sunscreen filter substance,
III) ≧15% and ≦70% by weight of at least one aliphatic alcohol having 1 to 4 carbon atoms,
IV) ≧0% and ≦20% by weight of at least one oil and/or ≧0% and ≦5% by weight of at least one wax, and
V) ≧0% and ≦40% by weight of further additives customary for sunscreen compositions,
based in each case on the total mass of the sunscreen composition, where components I) to V) add up to 100% by weight.

Component IV) is preferably equals zero only when component B) already contains at least one liquid oil-soluble sunscreen filter substance. The latter can then be regarded as an oil which is also able to dissolve further solid oil-soluble sunscreen filter substances.

The solids content of the polyurethane urea solution is preferably chosen such that the sunscreen composition contains preferably ≧1% and ≦10% by weight of the polyurethane urea as active substance, based on the total mass of the sunscreen composition.

The invention provides a sunscreen composition comprising at least one sunscreen filter substance and at least one polyurethane urea which has no ionically hydrophilizing groups and which is used dissolved in a solvent or solvent mixture, wherein the solvent consists of one or more monohydroxy-functional alcohols or a solvent mixture consisting of organic solvents and containing ≧50% by weight, based on the total mass of the solvent mixture, of at least one monohydroxy-functional alcohol is used.

The polyurethane urea used is formed from
a) at least one of an aliphatic, an araliphatic and a cycloaliphatic diisocyanate,
b) at least one polyether polyol having a number-average molecular weight Mₙ of ≧400 and ≦6000 g/mol and a hydroxyl functionality of ≧1.5 and ≦4,
c) at least one amino-functional compound having at least two isocyanate-reactive amino groups,
d) optionally, at least one alcohol having at least two hydroxyl groups and a molar mass of ≧60 and ≦399 g/mol,
e) optionally, at least one compound having a group reactive toward isocyanate groups and
f) optionally, ≦20% by weight, based on the total mass of the polyurethane urea, of at least one different polyol than b) having a number-average molecular weight Mₙ of ≧500 and ≦6000 g/mol and a hydroxyl functionality of ≧1.5 and ≦4.

In a first preferred embodiment of the sunscreen composition of the invention, the polyurethane urea does not have any hydrophilizing groups.

A second preferred embodiment of the invention comprises a sunscreen composition according to any of the abovementioned embodiments of the invention, characterized in that component b) is selected from poly(tetramethylene glycol) polyether polyols.

A third preferred embodiment of the invention comprises a sunscreen composition according to any of the abovementioned embodiments of the invention, characterized in that component b) has a number-average molecular weight Mₙ of ≧500 and ≦2500 g/mol and a hydroxyl functionality of ≧1.9 and ≦3.

A fourth preferred embodiment of the invention comprises a sunscreen composition according to any of the abovementioned embodiments of the invention, characterized in that component a) is selected from aliphatic, araliphatic and cycloaliphatic diisocyanates having at least one isocyanate group bonded to a tertiary carbon atom.

A fifth preferred embodiment of the invention comprises a sunscreen composition according to any of the abovementioned embodiments of the invention, characterized in that component a) is selected from IPDI and H12-MDI.

A sixth preferred embodiment of the invention comprises a sunscreen composition according to any of the abovementioned embodiments of the invention, characterized in that component c) is selected from amines having at least two amino groups bonded to primary and/or secondary carbon atoms.

A seventh preferred embodiment of the invention comprises a sunscreen composition according to any of the abovementioned embodiments of the invention, characterized in that component c) is selected from diamines of symmetric structure.

An eighth preferred embodiment of the invention comprises a sunscreen composition according to any of the abovementioned embodiments of the invention, characterized in that component c) is selected from ethylenediamine and H12-MDA.

A ninth preferred embodiment of the invention comprises a sunscreen composition according to any of the abovementioned embodiments of the invention, characterized in that the monohydroxy-functional alcohols are selected from aliphatic alcohols having 1 to 6 carbon atoms.

A tenth preferred embodiment of the invention comprises a sunscreen composition according to any of the abovementioned embodiments of the invention, characterized in that the sunscreen filter substances are present to an extent of ≧5% and ≦35% by weight, based on the total weight of the sunscreen composition.

An eleventh preferred embodiment of the invention comprises a sunscreen composition according to any of the abovementioned embodiments of the invention, characterized in that ≧4% and ≦12% by weight of the sunscreen filter substances are octocrylene.

A twelfth preferred embodiment of the invention comprises a sunscreen composition according to any of the abovementioned embodiments of the invention, characterized in that it comprises at least one oil-soluble sunscreen filter substance.

A thirteenth preferred embodiment of the invention comprises a sunscreen composition according to any of the abovementioned embodiments of the invention, characterized in that it comprises ≧5% and ≦20% by weight of oil-soluble sunscreen filter substance.

The present invention is elucidated by the following examples.

### EXAMPLES

Unless indicated otherwise, all percentages are based on weight. Unless stated otherwise, all analytical measurements relate to temperatures of 23° C. The solids contents (non-volatile component) were determined to DIN EN ISO 3251. Unless explicitly mentioned otherwise, NCO contents were determined by volumetric means to DIN EN ISO 11909. The check for free NCO groups was conducted by means of IR spectroscopy (band at 2260 cm⁻¹).

The viscosities reported were determined by means of rotary viscometry to DIN 53019 at 23° C. with a rotary viscometer from Anton Paar Germany GmbH, Ostfildern, DE. The number-average molecular weight was determined by gel permeation chromatography (GPC) in tetrahydrofuran at 23° C. The procedure is according to DIN 55672-1: “Gel permeation chromatography, Part 1—tetrahydrofuran as eluent” (SECurity GPC System from PSS Polymer Service, flow rate 1.0 ml/min; columns: 2×PSS SDV linear M, 8×300 mm, 5 μm; RID detector). Polystyrene samples of known molar mass are used for calibration. The number-average molecular weight is calculated with software support. Baseline points and evaluation limits are fixed in accordance with DIN 55672 Part 1.

Turbidity values [NTU] were determined by a scattered light measurement at a 90° angle (nephelometry) at a measurement radiation wavelength of 860 nm in accordance with DIN EN ISO 7027, conducted at 23° C. with a model 2100AN laboratory turbidimeter from HACH LANGE GmbH, Berlin, Germany.

### Substances and Abbreviations Used

POLYTHF 2000: polytetramethylene glycol polyol, OH number 56 mg KOH/g, number-average molecular weight 2000 g/mol (BASF AG, Ludwigshafen, DE)
POLYTHF 1000: polytetramethylene glycol polyol, OH number 112 mg KOH/g, number-average molecular weight 1000 g/mol (BASF AG, Ludwigshafen, DE)
Ethanol Unless stated otherwise, MEK-denatured ethanol from Nordbrand, Norhausen, DE was used.

Isocyanates and the further polymeric polyols were used from Covestro AG (formerly Bayer MaterialScience AG), Leverkusen, DE. Further chemicals were purchased from Sigma-Aldrich Chemie GmbH, Taufkirchen, DE. The raw materials, unless stated otherwise, were used without further purification or pretreatment.

### Example 1: Preparation of a Polyurethane Solution in Ethanol (Inventive)

150 g of POLYTHF 2000 and 37.50 g of POLYTHF 1000 were dewatered under membrane pump vacuum at 100° C. for one hour in a standard stirrer apparatus and then initially charged at 80° C. under nitrogen. Then 75.06 g of isophorone diisocyanate were added at 80° C. within 5 min and stirring at 110° C. was continued (about 3 hours) until the NCO value had gone below the theoretical value. The prepolymer was cooled to 40° C. and it was dissolved in 630.4 g of ethanol (denatured with diethyl phthalate) and then the temperature was reduced to 15° C. Then a solution of 37.6 g of methylenebis(4-aminocyclohexane) and 270 g of ethanol (denatured with diethyl phthalate) was metered in within 30 min; after a further 30 minutes at 20° C., isocyanate groups were still detectable by IR spectroscopy. Stirring of the mixture was continued at 23° C. for about 16 hours until no free isocyanate groups were detectable any longer by IR spectroscopy. The resultant clear, storage-stable solution had the following properties:

Solids content: 23%
Viscosity (viscometer, 23° C.): 280 mPas

### Example 2: Preparation of a Polyurethane Solution in Ethanol (Inventive)

300 g of POLYTHF 1000 were dewatered under membrane pump vacuum at 100° C. for one hour in a standard stirrer apparatus and then initially charged at 80° C. under nitrogen. Then 133.44 g of isophorone diisocyanate were added at 80° C. within 5 min and stirring at 110° C. was continued (about 3 hours) until the NCO value had gone below the theoretical value. The prepolymer was cooled to 40° C. and it was dissolved in 517 g of ethanol (denatured with MEK) and then the temperature was reduced to 16° C. Then a solution of 58.8 g of methylenebis(4-aminocyclohexane) and 222 g of ethanol (denatured with MEK) was metered in within 30 min; then a further 410 g of ethanol were added. Stirring was continued until no free isocyanate groups were detectable any longer by IR spectroscopy.

The resultant clear, storage-stable solution had the following properties:

Solids content: 30.2%
Viscosity (viscometer, 23° C.): 85000 mPas

### Example 3: Preparation of a Polyurethane Solution in Ethanol (Inventive)

211 g of POLYTHF 2000 and 52.7 g of POLYTHF 1000 were dewatered under membrane pump vacuum at 100° C. for one hour in a standard stirrer apparatus, then 5.4 g of neopentyl glycol were added and the mixture was subsequently initially charged at 80° C. under nitrogen. Then 93.4 g of isophorone diisocyanate were added at 80° C. within 5 min and stirring at 110° C. was continued (about 3 hours) until the NCO value had gone below the theoretical value. The prepolymer was cooled to 40° C. and it was dissolved in 420 g of ethanol (denatured with diethyl phthalate) and then the temperature was reduced to 17° C. Then a solution of 35.3 g of methylenebis(4-aminocyclohexane) and 180 g of ethanol (denatured with diethyl phthalate) was metered in within 30 min. A further 0.67 g of methylenebis(4-aminocyclohexane) were added and then stirring was continued until no free isocyanate groups were detectable any longer by IR spectroscopy.

The resultant clear, storage-stable solution had the following properties:

Solids content: 40.5%
Viscosity (viscometer, 23° C.): 7060 mPas

### Example 4: Preparation of a Polyurethane Solution in Ethanol (Inventive)

226.2 g of polypropylene glycol having a number-average molecular weight of 2000 g/mol and 62.5 g of polypropylene glycol having a number-average molecular weight of 1000 g/mol were dewatered under membrane pump vacuum at 100° C. for one hour in a standard stirrer apparatus, then the mixture was initially charged at 80° C. under nitrogen. Then 83.4 g of isophorone diisocyanate were added at 80° C. within 5 min and the mixture was stirred at 120° C. for 6 hours until the NCO value had gone below the theoretical value. The prepolymer was cooled to 40° C. and it was dissolved in 280 g of ethanol and then the temperature was reduced to 18° C. Then a solution of 34.1 g of methylenebis(4-aminocyclohexane) and 120 g of ethanol was metered in within 30 min. A further 4.5 g of methylenebis(4-aminocyclohexane) were added and then stirring was continued until no free isocyanate groups were detectable any longer by IR spectroscopy.

The resultant clear, storage-stable solution had the following properties:

Solids content: 49.8%
Viscosity (viscometer, 23° C.): 1100 mPas

### Example 5: Preparation of a Polyurethane Solution in Ethanol (Inventive)

160 g of POLYTHF 2000 and 40.0 g of POLYTHF 1000 were dewatered under membrane pump vacuum at 100° C. for one hour in a standard stirrer apparatus and then initially charged at 80° C. under nitrogen. Then 62.9 g of bis(4,4′-isocyanato-cyclohexyl)methane were added at 80° C. within 5 min and stirring at 110° C. was continued (about 3 hours) until the NCO value had gone below the theoretical value. The prepolymer was cooled to 40° C. and it was dissolved in 595 g of ethanol and then the temperature was reduced to 19° C. Then a solution of 20.2 g of methylenebis(4-aminocyclohexane) and 255 g of ethanol was metered in within 30 min. A further 4.5 g of methylenebis(4-aminocyclohexane) were added and then stirring was continued until no free isocyanate groups were detectable any longer by IR spectroscopy.

The resultant clear, storage-stable solution had the following properties:

Solids content: 25.2%
Viscosity (viscometer, 23° C.): 3400 mPas

### Comparative Example 1: Attempted Preparation of a Polyurethane Urea Solution in Ethanol

100 g of POLYTHF 2000, 25.0 g of POLYTHF 1000 and 127.5 g of a linear difunctional amorphous polyester diol based on adipic acid, hexane-1,6-diol and neopentyl glycol and having a number-average molecular weight of 1700 g/mol were dewatered under membrane pump vacuum at 100° C. for one hour in a standard stirrer apparatus and then initially charged at 80° C. under nitrogen. Then 66.7 g of isophorone diisocyanate were added at 80° C. within 5 min and stirring at 110° C. was continued (about 3 hours) until the NCO value had gone below the theoretical value. The prepolymer was cooled to 40° C. and it was dissolved in 720 g of ethanol, although the product did not dissolve completely, and then the temperature was reduced to 17° C. Then a solution of 25.2 g of methylenebis(4-aminocyclohexane) and 310 g of ethanol was metered in within 30 min, which gave rise to white turbidity. Then stirring was continued, which did not form a stable solution but resulted in a biphasic mixture from which the solid phase settled out.

### Comparative Example 2: Attempted Preparation of a Polyurethane Urea Solution in Ethanol

200 g of a linear difunctional polycarbonate diol based on hexane-1,6-diol, having a number-average molecular weight of 2000 g/mol, and 50 g of a linear difunctional polycarbonate diol based on hexane-1,6-diol, having a number-average molecular weight of 1000 g/mol, were dewatered under membrane pump vacuum at 100° C. for one hour in a standard stirrer apparatus and then initially charged at 80° C. under nitrogen. Then 66.7 g of isophorone diisocyanate were added at 80° C. within 5 min and stirring at 110° C. was continued (about 3 hours) until the NCO value had gone below the theoretical value. The prepolymer was cooled to 40° C. and it was dissolved in 720 g of ethanol, although the product did not dissolve completely, and then the temperature was reduced to 17° C. Then a solution of 25.2 g of methylenebis(4-aminocyclohexane) and 310 g of ethanol was metered in within 30 min, which gave rise to a biphasic mixture. Then stirring was continued, which did not form a stable solution but resulted in a biphasic mixture from which the solid phase settled out.

Compatibility with Ethanol

The compatibility of the polyurethane urea solutions prepared in accordance with the invention with ethanol compared to polyurethane-based film formers according to the prior art was tested. The polyurethanes that were used in the film formers according to prior art have ionically hydrophilizing groups. For this purpose, at 23° C., a mixture of ethanol and the film former was produced in each case, which contained 2% by weight of active substance (respective polyurethane), and the turbidity values of the mixtures were determined.

TABLE 1

| | | | Solids | | Turbidity |
|---|---|---|---|---|---|
| | | Supply | content | | value |
| Trade name | INCI name | form | [%] | Appearance | [NTU] |
| BAYCUSAN | Polyurethane-34 | Aqueous, | 40 | Milky, | 1410 |
| C 1000 | | ionically | | slight | |
| (Covestro AG, | | hydrophilizing | | sediment | |
| formerly | | polyurethane | | | |
| BMS AG) | | dispersion | | | |
| BAYCUSAN | Polyurethane-48 | Aqueous, | 30 | Slightly | 500 |
| C 1008 | | ionically | | bluish, | |
| (Covestro AG, | | hydrophilizing | | colloidal | |
| formerly | | polyurethane | | | |
| BMS AG) | | dispersion | | | |
| Inventive | — | Ethanolic | 23 | Clear | 3.5 |
| polyurethane | | solution | | | |
| urea | | | | | |
| solution | | | | | |
| AVALURE U 450 | PPG- | Aqueous, | 38 | Slightly | 102 |
| (Lubrizol) | 17/IPDI/DMPA | ionically | | bluish, | |
| | Copolymer | hydrophilizing | | colloidal | |
| | | polyurethane | | | |
| | | dispersion | | | |

### Performance-Related Comparative Tests:

Unless indicated otherwise, all percentages are based on weight.

The constituents specified in Table 2, by mixing the constituents, were used to produce a cosmetic composition having sunscreen properties. For this purpose, the components of the oil phase except for cyclopentasiloxane and caprylylmethicone were mixed and heated to 80° C. until the phase was clear. Subsequently, the oil phase was cooled down to 45° C. and cyclopentasiloxane and caprylylmethicone were added. The components of the ethanol phase were combined and stirred at 23° C. until a homogeneous mixture had formed. Then the ethanol phase was added to the oil phase and the two phases were stirred at 23° C. until a homogeneous mixture had formed.

For comparison, the same formulation was produced without the polyurethane urea solution of the invention.

For further comparison, the same composition was formulated, except that the polyurethane urea solution of the invention was replaced by other film formers such as DERMACRYL 79, LUVISKOL K-90, AVALURE U 450 or BAYCUSAN C1000 or C1008. All components were used here in the same amounts, and the amount of the film former component was chosen according to the concentration, such that the formulation contains 2% by weight of polymeric active substance.

TABLE 2

| Phase | Constituents | [% by wt.] |
|---|---|---|
| Ethanol phase | Ethanol | 47.70 |
| | Inventive ethanolic | 8.70 (2.00) |
| | polyurethane urea solution | |
| | (polyurethane urea | |
| | as active substance) | |
| | Butylene glycol | 2.50 |
| Oil phase | 4-(t-Butyl)-4'- | 4.00 |
| | methoxydibenzoylmethane | |
| | Homosalate | 7.00 |
| | Octocrylene | 7.00 |
| | Bis-Ethylhexyloxyphenol | 2.00 |
| | Methoxyphenyl | |
| | Triazin | |
| | Ethylhexyl Salicylate | 5.00 |
| | C₁₂₋₁₅ Alkyl Benzoate | 4.40 |
| | Dicaprylyl Carbonate | 5.80 |
| | Tridecyl Salicylate | 2.90 |
| | Cyclopentasiloxane | 1.50 |
| | Caprylylmethicone | 1.50 |
| | Sum total | 100.00 |

Table 3 shows the properties of the cosmetic compositions obtained.

TABLE 3

| | | | Solids | | Turbidity |
|---|---|---|---|---|---|
| | INCI | Supply | content | Formulation | value |
| Trade name | name | form | [%] | appearance | [NTU] |
| BAYCUSAN | Polyurethane-34 | Aqueous PUD | 40 | Milky | Not |
| C 1000 | | | | | measurable |
| (Covestro AG, | | | | | |
| formerly | | | | | |
| Bayer | | | | | |
| MaterialScience | | | | | |
| AG) | | | | | |
| BAYCUSAN | Polyurethane-48 | Aqueous PUD | 30 | Milky | Not |
| C 1008 | | | | | measurable |
| (Covestro AG, | | | | | |
| formerly | | | | | |
| Bayer | | | | | |
| MaterialScience | | | | | |
| AG) | | | | | |
| Inventive | — | Ethanolic | 23 | Clear | 3.5 |
| polyurethane | | solution | | | |
| urea solution | | | | | |
| AVALURE | PPG-17/IPDI/ | Aqueous PUD | 38 | Unstable | — |
| U 450 | DMPA | | | | |
| (Lubrizol) | copolymer | | | | |
| LUVISKOL | PVP | Solid | 100 | Unstable | — |
| K 90 | | | | | |
| (BASF) | | | | | |
| DERMACRYL 79 | Acrylate/octyl- | Solid | 100 | Clear | 3.9 |
| (AkzoNobel) | acrylamide | | | | |
| | copolymer | | | | |

### Results of the In Vivo SPF Measurement

For the clear formulations, the SPF values were determined in vivo. The results are compiled in Table 3. The values determined clearly show the high SPF-boosting effect of the sunscreen composition of the invention.

The in vivo measurement of the SPF was made according to ISO 24444:2010 on 5 test subjects.

TABLE 4

| SPF | SPF with | SPF with |
|---|---|---|
| without | inventive | acrylate/octylacryl- |
| polyurethane | polyurethane | amide copolymer |
| solution | urea solution | DERMACRYL 79 |
| 11.4 | 31.2 | 24.1 |

This specification has been written with reference to various non-limiting and non-exhaustive embodiments. However, it will be recognized by persons having ordinary skill in the art that various substitutions, modifications, or combinations of any of the disclosed embodiments (or portions thereof) may be made within the scope of this specification. Thus, it is contemplated and understood that this specification supports additional embodiments not expressly set forth herein. Such embodiments may be obtained, for example, by combining, modifying, or reorganizing any of the disclosed steps, components, elements, features, aspects, characteristics, limitations, and the like, of the various non-limiting embodiments described in this specification. In this manner, Applicant(s) reserve the right to amend the claims during prosecution to add features as variously described in this specification, and such amendments comply with the requirements of 35 U.S.C. §112(a), and 35 U.S.C. §132(a).

Various aspects of the subject matter described herein are set out in the following numbered clauses:

1. A process for producing a cosmetic composition, characterized in that at least one polyurethaneurea which has no ionically hydrophilizing groups and has been dissolved in a solvent or solvent mixture is used, the solvent consisting of one or more monohydroxy-functional alcohols or being a solvent mixture consisting of organic solvents and containing ≧50% by weight, based on the total mass of the solvent mixture, of at least one monohydroxy-functional alcohol, wherein the polyurethane urea has been formed from a) at least one aliphatic, araliphatic and/or cycloaliphatic diisocyanate, b) at least one polyether polyol having a number-average molecular weight Mn of ≧400 and ≦6000 g/mol and a hydroxyl functionality of ≧1.5 and ≦4, c) at least one amino-functional compound having at least two isocyanate-reactive amino groups, d) optionally, at least one alcohol having at least two hydroxyl groups and a molar mass of ≧60 and ≦399 g/mol, e) optionally, at least one compound having a group reactive toward isocyanate groups and f) optionally, ≦20% by weight, based on the total mass of the polyurethaneurea, of at least one different polyol than b) having a number-average molecular weight Mn of ≧500 and ≦6000 g/mol and a hydroxyl functionality of ≧1.5 and ≦4.
2. The process as in clause 1, characterized in that the polyurethane urea does not have any hydrophilizing groups.
3. The process as in clause 1 or 2, characterized in that component b) is selected from poly(tetramethylene glycol) polyether polyols.
4. The process as in any of clauses 1 to 3, characterized in that component b) has a number-average molecular weight Mn of ≧500 and ≦2500 g/mol and a hydroxyl functionality of ≧1.9 and ≦3.
5. The process as in any of clauses 1 to 4, characterized in that component a) is selected from aliphatic, araliphatic and cycloaliphatic diisocyanates having at least one isocyanate group bonded to a secondary and/or tertiary carbon atom.
6. The process as in any of clauses 1 to 5, characterized in that component a) is selected from IPDI and/or H12-MDI.
7. The process as in any of clauses 1 to 6, characterized in that component c) is selected from amines having at least two amino groups bonded to primary and/or secondary carbon atoms.
8. The process as in any of clauses 1 to 7, characterized in that component c) is selected from diamines of symmetric structure.
9. The process as in any of clauses 1 to 8, characterized in that component c) is selected from ethylenediamine and/or H12-MDA.
10. The process as in any of clauses 1 to 9, characterized in that the monohydroxy-functional alcohols are selected from aliphatic alcohols having one to six carbon atoms.
11. The process as in any of clauses 1 to 10, characterized in that the cosmetic composition comprises at least one oil-soluble sunscreen filter substance.
12. A cosmetic composition obtainable by a process as in any of clauses 1 to 12.
13. A process for producing a cosmetic composition on skin, nails and/or keratinic fibers using cosmetic compositions as in clause 12, wherein the cosmetic composition is applied to skin, nails and/or keratinic fibers.
14. A sunscreen composition comprising at least one sunscreen filter substance and at least one polyurethaneurea which has no ionically hydrophilizing groups and has been dissolved in a solvent or solvent mixture, wherein the solvent consists of one or more monohydroxy-functional alcohols or is a solvent mixture consisting of organic solvents and containing ≧50% by weight, based on the total mass of the solvent mixture, of at least one monohydroxy-functional alcohol, wherein the polyurethaneurea has been formed from a) at least one aliphatic, araliphatic and/or cycloaliphatic diisocyanate, b) at least one polyether polyol having a number-average molecular weight Mₙ of ≧400 and ≦6000 g/mol and a hydroxyl functionality of ≧1.5 and ≦4, c) at least one amino-functional compound having at least two isocyanate-reactive amino groups, d) optionally, at least one alcohol having at least two hydroxyl groups and a molar mass of ≧60 and ≦399 g/mol, e) optionally, at least one compound having a group reactive toward isocyanate groups and f) optionally, ≦20% by weight, based on the total mass of the polyurethaneurea, of at least one different polyol than b) having a number-average molecular weight Mₙ of ≧500 and ≦6000 g/mol and a hydroxyl functionality of ≧1.5 and ≦4.
15. The sunscreen composition as in clause 14 for protection of skin and/or hair from adverse effects of solar radiation.

1: A process for producing a cosmetic composition, comprising at least one polyurethane urea formed from
a) at least one of an aliphatic, an araliphatic and a cycloaliphatic diisocyanate,
b) at least one polyether polyol having a number-average molecular weight Mₙ of ≧400 and ≦6000 g/mol and a hydroxyl functionality of ≧1.5 and ≦4,
c) at least one amino-functional compound having at least two isocyanate-reactive amino groups,
d) optionally, at least one alcohol having at least two hydroxyl groups and a molar mass of ≧60 and ≦399 g/mol,
e) optionally, at least one compound having a group reactive toward isocyanate groups and
f) optionally, ≦20% by weight, based on the total mass of the polyurethane urea, of at least one different polyol than b) having a number-average molecular weight Mₙ of ≧500 and ≦6000 g/mol and a hydroxyl functionality of ≧1.5 and ≦4,
wherein the polyurethane urea has no ionically hydrophilizing groups and is dissolved in a solvent or solvent mixture, wherein the solvent consists of one or more monohydroxy-functional alcohols or is a solvent mixture consisting of organic solvents and containing ≧50% by weight, based on the total mass of the solvent mixture, of at least one monohydroxy-functional alcohol.

2: The process according to claim 1, wherein the polyurethane urea contains no hydrophilizing groups.

3: The process according to claim 1, wherein component b) is a poly(tetramethylene glycol) polyether polyol.

4: The process according to claim 1, wherein component b) has a number-average molecular weight Mₙ of ≧500 and ≦2500 g/mol and a hydroxyl functionality of ≧1.9 and ≦3.

5: The process according to claim 1, wherein component a) is selected from the group consisting of aliphatic, araliphatic and cycloaliphatic diisocyanates having at least one isocyanate group bonded to a secondary or tertiary carbon atom.

6: The process according to claim 1, wherein component a) is selected from the group consisting of IPDI and H12-MDI.

7: The process according to claim 1, wherein component c) is selected from the group consisting of amines having at least two amino groups bonded to primary or secondary carbon atoms.

8: The process according to claim 1, wherein component c) is a diamine of symmetric structure.

9: The process according to claim 1, wherein component c) is selected from the group consisting of ethylenediamine and H12-MDA.

10: The process according to claim 1, wherein the monohydroxy-functional alcohol is selected from an aliphatic alcohol having one to six carbon atoms.

11: The process according to claim 1, wherein the cosmetic composition further comprises at least one oil-soluble sunscreen filter substance.

12: A cosmetic composition produced by the process according to claim 1.

13: A process comprising applying a cosmetic composition produced by the process according to claim 1 to one of skin, nails and keratinic fibers.

14: A sunscreen composition comprising at least one sunscreen filter substance and at least one polyurethane urea formed from
a) at least one of an aliphatic, an araliphatic and a cycloaliphatic diisocyanate,
b) at least one polyether polyol having a number-average molecular weight Mₙ of ≧400 and ≦6000 g/mol and a hydroxyl functionality of ≧1.5 and ≦4,
c) at least one amino-functional compound having at least two isocyanate-reactive amino groups,
d) optionally, at least one alcohol having at least two hydroxyl groups and a molar mass of ≧60 and ≦399 g/mol,
e) optionally, at least one compound having a group reactive toward isocyanate groups and
f) optionally, ≦20% by weight, based on the total mass of the polyurethane urea, of at least one different polyol than b) having a number-average molecular weight Mₙ of ≧500 and ≦6000 g/mol and a hydroxyl functionality of ≧1.5 and ≦4
wherein the polyurethane urea contains no ionically hydrophilizing groups and is dissolved in a solvent or solvent mixture, wherein the solvent consists of one or more monohydroxy-functional alcohols or is a solvent mixture consisting of organic solvents and containing ≧50% by weight, based on the total mass of the solvent mixture, of at least one monohydroxy-functional alcohol.

15: A method for protection of one of skin and hair comprising applying the sunscreen according to claim 14.
